SEQUENCE LISTING (22 sequences)

SEQ ID NO: 1 - protein, 8 aa, Artificial Sequence
  >CDR1 HC VH MN
GYTFTSYW

SEQ ID NO: 2 - protein, 8 aa, Artificial Sequence
  >CDR2 HC VH MN
IHPNSDNT

SEQ ID NO: 3 - protein, 13 aa, Artificial Sequence
  >CDR3 HC VH MN
AIIITPVVPKFDY

SEQ ID NO: 4 - protein, 6 aa, Artificial Sequence
  >CDR1 LC VH MN
HDVGTS

SEQ ID NO: 5 - protein, 3 aa, Artificial Sequence
  >CDR2 LC VH MN
WAS

SEQ ID NO: 6 - protein, 9 aa, Artificial Sequence
  >CDR3 LC VH MN
QQYSSYPLT

SEQ ID NO: 7 - protein, 120 aa, Artificial Sequence
  >VR HC VH-MN (parental mouse)
QVQLQQPGAELVKPGASMKLSCKASGYTFTSYWMHWVKQRPGQGLEWIGMIHPNSDNTIYYEKFKSKATLTVDKSSSTAYMQLSSLTSEDSAVYYCAIIITPVVPKFDYWGQGTTLTVSS

SEQ ID NO: 8 - protein, 107 aa, Artificial Sequence
  >VR LC VH-MN (parental mouse)
DIVMTQSHQFMSTSVGDRVSVTCKASHDVGTSVAWYQQKPGQSPKLLIYWASTRHTGVPDRFTGSGSGTDFTLTISNVQSEDLADYFCQQYSSYPLTFGAGTKLELK

SEQ ID NO: 9 - protein, 120 aa, Artificial Sequence
  >VR HC VH-MN clone 3-1 (humanized)
QVQLQQSGAELVKPGASMKLSCKASGYTFTSYWMHWVRQAPGQGLEWIGAIHPNSDNTIYYQKFKSRVTLTVDKSISTAYMQLSSLTSEDTAVYYCAIIITPVVPKFDYWGQGTTVTVSS

SEQ ID NO: 10 - protein, 107 aa, Artificial Sequence
  >VR LC VH-MN clone 3-1 (humanized)
DIQMTQSPSFLSASVGDRVTITCRASHDVGTSLAWYQQKPGQAPKLLIYWASTLQTGVPSRFSGSGSGTEFTLTISSLQPEDFATYYCQQYSSYPLTFGPGTTVDMK

SEQ ID NO: 11 - protein, 120 aa, Artificial Sequence
  >VR HC VH-MN clone 5-2 (humanized)
QVQLQQSGAELVKPGASLKLSCKASGYTFTSYWMHWVRQAPGQGLEWIGAIHPNSDNTIYYQKFKSRFTITVDKSTSTAYMQLSSLTSEDTAVYYCAIIITPVVPKFDYWGQGTTVTVSS

SEQ ID NO: 12 - protein, 107 aa, Artificial Sequence
  >VR LC VH-MN clone 5-2 (humanized)
DIVMTQSSSFMSASVGDRVSITCKASHDVGTSLAWYQQKPGQSPKLLIYWASTRQTGVPDRFSGSGSGTDFTLTISSVQSEDVATYFCQQYSSYPLTFGQGTKLEIK

SEQ ID NO: 13 - protein, 120 aa, Artificial Sequence; identical to SEQ ID NO: 11
  >VR HC VH-MN clone 5-3 (humanized)

SEQ ID NO: 14 - protein, 107 aa, Artificial Sequence
  >VR LC VH-MN clone 5-3 (humanized)
DIVMTQSPDSLAVSLGERATITCKSSHDVGTSLAWYQQKPGQPPKLLIYWASTRESGVPDRFSGSGSGTDFTLTISSLQAEDVAVYYCQQYSSYPLTFGQGTKLEIK

SEQ ID NO: 15 - DNA, 27 bp, Artificial Sequence
  >forward primer univ-Not1-SP-F
gaggaggcggccgccatgaactttggg

SEQ ID NO: 16 - DNA, 21 bp, Artificial Sequence
  >reverse primers huCG1-B
aagaccgatgggcccttggtg

SEQ ID NO: 17 - DNA, 21 bp, Artificial Sequence
  >IgHV and huCK-B
gaagacagatggtgcagccac

SEQ ID NO: 18 - DNA, 20 bp, Artificial Sequence
  >primer pPBseq13
ggccagcttggcacttgatg

SEQ ID NO: 19 - DNA, 20 bp, Artificial Sequence
  >EF1aNotI_F
ccatttcaggtgtcgtgagc

SEQ ID NO: 20 - DNA, 20 bp, Artificial Sequence
  >CG-revseq-1
gttcggggaagtagtccttg

SEQ ID NO: 21 - DNA, 28 bp, Artificial Sequence
  >Intron-rev-1
gtggatgtcagtaagaccaataggtgcc

SEQ ID NO: 22 - DNA, 21 bp, Artificial Sequence
  >CMVseq2
gcagtgtagtctgagcagtac

(19) United States
(12) Patent Application Publication
(10) Pub. No.: US 2019/0112385 A1
(43) Pub. Date: Apr. 18, 2019

(54) ANTI-MESOTHELIN ANTIBODIES

(71) Applicant: NBE-THERAPEUTICS AG, Basel (CH)

(72) Inventors: Ina HELLMANN, Basel (CH); Lorenz WALDMEIER, Basel (CH); Ulf GRAWUNDER, Hersberg (CH); Roger BEERLI, Adlikon bei Regensdorf (CH)

(73) Assignee: NBE-THERAPEUTICS AG, Basel (CH)

(21) Appl. No.: 15/768,231

(22) PCT Filed: Oct. 31, 2016
(86) PCT No.: PCT/EP2016/076255

§ 371 Date: Nov. 23, 2018

(30) Foreign Application Priority Data
Oct. 30, 2015 (EP) 15192436.2

(51) Int. Cl. C07K 16/32; A61K 47/68; C07K 16/30; A61P 35/00; C07K 14/00

(52) U.S. Cl. CPC C07K 16/32; A61K 47/6803; A61K 47/6851; C07K 14/003; C07K 16/3023; A61P 35/00; A61K 47/6889

(57) ABSTRACT

The present invention relates to a human or humanized antibody, or an antibody-based binding protein, modified antibody format retaining target binding capacity, antibody derivative or fragment retaining target binding capacity, which targets Mesothelin (MN). It further relates to bi- or multispecific antibodies, to Immunoligand-Drug Conjugates, to Chimeric Antigen Receptors and to T-cells comprising such Chimeric Antigen Receptors.

### CROSS-REFERENCE TO RELATED APPLICATIONS

This application is a 35 U.S.C. § 371 filing of International Patent Application No. PCT/EP2016/076255, filed Oct. 31, 2016, which claims priority to European Patent Application No. 15192436.2, filed Oct. 30, 2015, the entire contents of which are incorporated herein by reference.

The present invention relates to anti-Mesothelin antibodies, including bispecific and multispecific antibodies, Immunoligand-Toxin Conjugates targeting Mesothelin, and anti Mesothelin CARs and CAR cells.

### INTRODUCTION

For several neoplastic diseases, no efficacious therapeutic approaches exist. Pancreatic adenocarcinoma, even if diagnosed early, often has a poor prognosis. Mesothelioma also has a very poor one-year survival rate. Lung cancer is one of the most frequent cancers and often diagnosed at a late stage leading to a poor five-year survival rate of only 15%.

It is an object of the present invention to provide new therapeutic approaches to address these diseases.

### PREFERRED EMBODIMENTS

It is to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that this does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

According to a first embodiment of the invention, a human or humanized antibody, or an antibody-based binding protein, modified antibody format retaining target binding capacity, or an antibody derivative or fragment retaining target binding capacity, is provided, which targets Mesothelin (MN).

In a second embodiment also an Immunoligand-Drug conjugate of said first embodiment with a functional moiety covalently coupled to a human or humanized antibody, or an antibody-based binding protein, or a modified antibody format retaining target binding capacity, or an antibody derivative or fragment retaining target binding capacity is provided, which targets Mesothelin (MN). This conjugate is preferably an antibody drug conjugate (ADC), to which preferably a small molecular weight cellular toxin is conjugated, preferably site-specifically, and preferably by, but not limited to sortase-enzyme mediated conjugation technology (SMAC-technology) disclosed in WO2014140317.

In a third embodiment, also mammalian cells carrying receptors comprising a human or humanized antibody, or an antibody-based binding protein, or a modified antibody format retaining target binding capacity, or an antibody derivative or fragment retaining target binding capacity for Mesothelin (MN) is provided. Such mammalian cells are preferably T cells of the immune system, carrying preferably chimeric antigen receptors (CARs) comprising said human or humanized antibody, or an antibody-based binding protein, or a modified antibody format retaining target binding capacity, or an antibody derivative or fragment retaining target binding capacity for Mesothelin (MN). In a further preferred embodiment, these mammalian cells are therefore CAR T cells comprising said human or humanized antibody, or an antibody-based binding protein, or a modified antibody format retaining target binding capacity, or an antibody derivative or fragment retaining target binding capacity for Mesothelin (MN).

Mesothelin (MN) is a GPI-anchored glycoprotein that is present on normal mesothelial cells lining the pleura, pericardium and peritoneum. In contrast to this limited distribution on normal tissue, mesothelin is highly expressed on the surface of tumor cells from diverse origins, including mesothelioma (nearly 100%), pancreatic adenocarcinomas (nearly 100%), ovarian carcinoma (70%), lung adenocarcinoma (50%) as well as cholangiocarcinomas (30%). It is further expressed in some types of Breast cancer, in particular triple negative breast cancer.

The inventors have surprisingly found that Mesothelin provides a promising target for cancer therapy, in particular of the diseases set forth above, e.g., Pancreatic adenocarcinoma, Mesothelioma, and/or Lung cancer.

Although widely known, no mesothelin-specific antibodies have so far been approved for cancer therapy. To be applicable for such use, antibodies targeting Mesothelin, and Immunoligand-Toxin-Conjugates targeting Mesothelin, should show minimal immuogenicity, thus minimizing the possibility to be cleared from the system by the immune-system of patients and thus resulting in prolonged serum half-life and, consequently, higher efficiency. Further, reduced immunogenicity avoids unwanted and sometimes even life threatening side effects, like immunogenic reactions.

Therefore the present invention provides human and humanized anti-mesothelin antibodies, which are expected to result in minimal immunogenicity and with efficacy in the treatment of neoplastic conditions.

In one specific embodiment, humanized anti-Mesothelin antibodies, or antibody-based binding proteins, modified antibody formats retaining target binding capacity, antibody derivatives or fragments retaining target binding capacity are provided.

The term “humanized antibody” refers to a chimeric antibody that contains sequences derived from human and non-human (e.g., rabbit) immunoglobulins such that substantially all of the CDR regions are of non-human origin, while substantially all of the FR regions correspond to those of a human immunoglobulin sequence.

In one embodiment, the antibody has been humanized from a rodent or rabbit parent antibody, i.e., comprises CDR regions that are of rodent or rabbit origin.

In a preferred embodiment, the antibody comprises at least the 3 CDR sequences:

SEQ ID No 1
CDR1 HC
SEQ ID No 2
CDR2 HC
SEQ ID No 3
CDR3 HC

In another preferred embodiment, the antibody comprises at least the 3 CDR sequences:

SEQ ID No 4
CDR1 LC
SEQ ID No 5
CDR2 LC
SEQ ID No 6
CDR3 LC

In both cases, it is to be understood that the definition of the CDR (“Complementarity Determining Region”) is based on the “IMGT unique numbering for all IG and TR V-REGIONs of all species: interest for structure and evolution”. Further, “CDR LC” means Light Chain CDR, while “CDR HC” means Heavy Chain CDR.

In a preferred embodiment, the antibody comprises at least one heavy chain or light chain variable region sequence that is 95% identical, preferably 96 or even 97% identical, more preferably 98% or even 99% identical, and most preferably 100% to a sequence selected from the group consisting of:

SEQ ID NO 9
VR HC
SEQ ID NO 10
VR LC
SEQ ID NO 11
VR HC
SEQ ID NO 12
VR LC
SEQ ID NO 13
VR HC
SEQ ID NO 14
VR LC

“VR HC” means Heavy Chain Variable Sequence, while “VR LC” means Light Chain Variable Sequence.

In a preferred embodiment, the antibody is humanized from
murine anti Mesothelin antibody VH-MN
   and/or is selected from the group consisting of
VH-MN clone 3-1 (humanized), also called huMN3-1
VH-MN clone 5-2 (humanized), also called huMN5-2
VH-MN clone 5-3 (humanized), also called huMN5-2
   and/or antibodies sharing at least 95%, preferably 96 or even 97% identical, more preferably 98% or even 99% identical, and most preferably 100% amino acid sequence identify with any of the antibodies mentioned above.

As can be seen, the variable region sequences were taken from a murine anti Mesothelin antibody, and humanized by mutation of the variable regions in framework regions (which are not directly involved in binding), towards a more human-like sequence (humanization). Sequences directly involved in binding were left unchanged (CDR-grafting approach).

Humanization of framework-regions was achieved by first engineering whole IgG antibody-coding variable-region libraries that contained different version of humanized variable regions (47 sequence variants for each chain), which were then screened for maximal mesothelin-binding using a state-of-the art mammalian antibody surface-display technology (“Transpo-mAb”, disclosed in WO2014013026A1, the content of which is incorporated by reference herein) in order to preserve the favorable characteristics of the original antibodies, which otherwise are easily lost upon said sequence manipulations due to changes in antibody structure.

In a preferred embodiment, the antibody has as at least one of the characteristics set forth in table 1.

According to yet another embodiment of the invention, a human or humanized antibody is provided, or an antibody-based binding protein, modified antibody format retaining target binding capacity, antibody derivative or fragment retaining target binding capacity, which
(i) has a binding affinity for Mesothelin (MN) that is at least as high or substantially as high as the binding affinity of an antibody, antibody-based binding protein, modified antibody format, antibody derivative or fragment according to any of claims 1-6, and/or
(ii) competes with an antibody, antibody-based binding protein, modified antibody format, antibody derivative or fragment according to any of claims 1-7 for binding to Mesothelin (MN).

In one embodiment of the antibody, antibody-based binding protein, modified antibody format, antibody derivative or fragment of any of the aforementioned claims, the Mesothelin (MN) is human MN.

According to another embodiment of the invention, the antibody-based binding protein, modified antibody format, antibody derivative or fragment of any of the aforementioned claims is a bispecific antibody or a multispecific antibody.

The terms “bispecific antibody” and “multispecific antibody” refers to an antibody having the capacity to bind to two, or more, distinct epitopes either on a single antigen or two different antigens, out of which one is ROR1. Bispecific antibodies of the present invention can be produced via biological methods, such as somatic hybridization; or genetic methods, such as the expression of a non-native DNA sequence encoding the desired antibody structure in an organism; chemical methods, such as chemical conjugation of two antibodies; or a combination thereof (Kontermann, R. E. In: Bispecific Antibodies. Kontermann R E (ed.), Springer Heidelberg Dordrecht London New York, pp. 1-28 (2011)).

Chemically conjugated bispecific antibodies arise from the chemical coupling of two existing antibodies or antibody fragments. Typical couplings include cross-linking two different full-length antibodies, cross-linking two different Fab′ fragments to produce a bispecific F(ab′)2, and cross-linking a F(ab′)2 fragment with a different Fab′ fragment to produce a bispecific F(ab′)3. For chemical conjugation, oxidative reassociation strategies can be used. Current methodologies include the use of the homo- or heterobifunctional cross-linking reagents (Id.). Heterobifunctional cross-linking reagents have reactivity toward two distinct reactive groups on, for example, antibody molecules. Examples of heterobifunctional cross-linking reagents include SPDP (N-succinimidyl-3-(2-pyridyldithio)propionate), SATA (succinimidyl acetylthioacetate), SMCC (succinimidyl trans-4-(maleimidylmethyl) cyclohexane-1-carboxylate), EDAC (1-ethyl-3-(3-dimethylaminopropyl) carbodiimide), PEAS (N-((2-pyridyldithio)ethyl)-4-azidosalicylamide), ATFB, SE (4-azido-2,3,5,6-tetrafluorobenzoic acid, succinimidyl ester), benzophenone-4-maleimide, benzophenone-4-isothiocyanate, 4-benzoylbenzoic acid, succinimidyl ester, iodoacetamide azide, iodoacetamide alkyne, Click-iT maleimide DIBO alkyne, azido (PEO)4 propionic acid, succinimidyl ester, alkyne, succinimidyl ester, Click-iT succinimidyl ester DIBO alkyne, Sulfo—SBED (Sulfo-N-hydroxysuccinimidyl-2-(6-[biotinamido]-2-(p-azido benzamido)-hexanoamido) ethyl-1,3′-dithioproprionate), photoreactive amino acids {e.g., L-Photo-Leucine and L-Photo-Methionine), NHS-haloacetyl crosslinkers such as, for example, Sulfo-SIAB, SIAB, SBAP, SIA, NHS—maleimide crosslinkers such as, for example, Sulfo-SMCC, SM(PEG)n series crosslinkers, SMCC, LC-SMCC, Sulfo-EMCS, EMCS, Sulfo-GMBS, GMBS, Sulfo-KMUS, Sulfo-MBS, MBS, Sulfo-SMPB, SMPB, AMAS, BMPS, SMPH, PEG12-SPDP, PEG4-SPDP, Sulfo-LC-SPDP, LC-SPDP, SMPT, DCC (N, N′-Dicyclohexylcarbodiimide), EDC (1-Ethyl-3-(3-dimethylaminopropyl) carbodiimide), NHS (N-hydroxysuccinimide), Sulfo-NHS (N-hydroxysulfosuccinimide), BMPH, EMCH, KMUH, MPBH, PDPH, and PMPI.

Homobifunctional cross-linking reagents have reactivity toward the same reactive group on a molecule, for example, an antibody. Examples of homobifunctional cross-linking reagents include DTNB (5,5′-dithiobis(2-nitrobenzoic acid), o-PDM (o-phenylenedimaleimide), DMA (dimethyl adipimidate), DMP (dimethyl pimelimidate), DMS (dimethyl suberimidate), DTBP (dithiobispropionimidate), BS(PEG)5, BS(PEG)9, BS3, BSOCOES, DSG, DSP, DSS, DST, DTSSP, EGS, Sulfo-EGS, TSAT, DFDNB, BM(PEG)n crosslinkers, BMB, BMDB, BMH, BMOE, DTME, and TMEA.

Somatic hybridization is the fusion of two distinct hybridoma (a fusion of B cells that produce a specific antibody and myeloma cells) cell lines, producing a quadroma capable of generating two different antibody heavy (VHA and VHB) and light chains (VLA and VLB). (Kontermann, R. E. In: Bispecific Antibodies. Kontermann R E (ed.), Springer Heidelberg Dordrecht London New York, pp. 1-28 (2011)). These heavy and light chains combine randomly within the cell, resulting in bispecific antibodies (a VHA combined with a VLA and a VHB combined with a VLB), as well as some nonfunctional (e.g. two VHAs combined with two VLBs) and monospecific (two VHAs combined with two VLAs) antibodies. The bispecific antibodies can then be purified using, for example, two different affinity chromatography columns. Similar to monospecific antibodies, bispecific antibodies may also contain an Fc region that elicits Fc-mediated effects downstream of antigen binding. These effects may be reduced by, for example, proteolytically cleaving the Fc region from the bispecific antibody by pepsin digestion, resulting in bispecific F(ab′)2 molecules (Id.).

Bispecific antibodies may also be generated via genetic means, e.g., in vitro expression of a plasmid containing a DNA sequence corresponding to the desired antibody structure. See, e.g., Kontermann, R. E. In: Bispecific Antibodies. Kontermann R E (ed.), Springer Heidelberg Dordrecht London New York, pp. 1-28 (2011). Such bispecific antibodies are discussed in greater detail below.

A bispecific antibody of the present invention may be bivalent, trivalent, or tetravalent. As used herein, “valent”, “valence”, “valencies”, or other grammatical variations thereof, mean the number of antigen binding sites in an antibody molecule.

Further provided are
a) an isolated nucleic acid sequence, or a set thereof, that encodes an antibody, antibody-based binding protein, modified antibody format, antibody derivative or fragment according to the above description, or a bispecific antibody or a multispecific antibody according to the above description,
b) a vector comprising at least one such nucleic acid sequence,
c) an isolated cell expressing an antibody, antibody-based binding protein, modified antibody format, antibody derivative or fragment according to the above description, or a bispecific antibody or a multispecific antibody according to the above description,
d) and/or comprising a nucleic acid sequence, or a set thereof, according to the above description, or a vector according to the above description, and
e) a method of producing an antibody, antibody-based binding protein, modified antibody format, antibody derivative or fragment according to the above description, comprising culturing of a cell according to the above description, and purification of the antibody, antibody-based binding protein, modified antibody format, antibody derivative or fragment.

According to another aspect of the invention, an Immunoligand-Drug Conjugate having the general formula A-(L)n-(T)n is provided, in which
A is an Immunoligand targeting Mesothelin,
L is a linker,
T is a toxin
   and in which n and m are integers between ≥1 and ≤10.

In this construct, (L)n can mean several linkers which form a unitary chain that conjugates one toxin to the one Immunoligand, and/or several linkers which connect several toxins to the one Immunoligand. Likewise, (L)n can mean several linkers which conjugate two Subdomains of the same Immunologand to two toxin molecules.

| | | Several linkers which |
|---|---|---|
| | | conjugate two subdomains |
| Several linkers | Several linkers which | (SD1, SD2) of the same |
| which form | connect several toxins to | Immunoligand to two toxin |
| a unitary chain | the one Immunoligand. | molecules |
| A-L₁-[ . . . ]-Lₙ-T | T-L₁-A-L₁-T | A_{SD1}-L₁-T |
| | | A_{SD2}-L₁-T |

The resulting Immunoligand-Toxin-Conjugate would thus have a Toxin/Immunoligand ratio of ≥1 and ≤10. Preferably, n and m are integers between ≥1 and ≤4. The resulting Immunoligand-Toxin-Conjugate would thus have an Toxin/Immunoligand ratio of ≥1 and ≤4.

As used herein, the term “immunoligand” is meant to define an entity, an agent or a molecule that has affinity to a given target, e.g., a receptor, a cell surface protein, a cytokine or the like. Such Immunoligand may optionally block or dampen agonist-mediated responses, or inhibit receptor-agonist interaction. Most importantly, however, the immunoligand may serve as a shuttle to deliver a payload to a specific site, which is defined by the target recognized by said immunoligand. Thus, an Immunoligand targeting a receptor, delivers its payload to a site which is characterized by abundance of said receptor.

In a preferred embodiment, the Immunoligand is at least one selected from the group consisting of an
antibody
antibody-based binding protein
modified antibody format retaining target binding capacity,
antibody derivative or fragment retaining target binding capacity, and/or
bispecific antibody or a multispecific antibody.
antibody mimetic, and/or
aptamer

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid antibody, which comprises only a heavy chain, single domain antibodies (dAbs) which can either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype.

An “antibody-based binding protein”, as used herein, may represent any protein that contains at least one antibody-derived V_{H}, V_{L}, or C_{H} immunoglobulin domain in the context of other non-immunoglobulin, or non-antibody derived components. Such antibody-based proteins include, but are not limited to (i) Fₑ-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

An “antibody drug conjugate” (ADC), as used herein, relates to either an antibody, or an antibody fragment, or an antibody-based binding protein, coupled to a small molecular weight active pharmaceutical ingredient (API), including, but not limited to a toxin (including e.g., but not limited to, tubulin inhibitors, actin binders, RNA polymerase inhibitors, DNA-intercalating and modifying/damaging drugs), a kinase inhibitor, or any API that interferes with a particular cellular pathway that is essential for the survival of a cell and/or essential for a particular physiologic cellular pathway.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (V_{L}), variable heavy (V_{H}), constant light (C_{L}) and constant heavy 1 (C_{H}I) domains; (ii) a F(ab′)2 fragment, which is a bivalent fragment comprising two Fab fragments linked by a disulfide bridge at the hinge region; (iii) a heavy chain portion of a F_{ab} (Fa) fragment, which consists of the V_{H} and C_{H}I domains; (iv) a variable fragment (Fᵥ) fragment, which consists of the V_{L} and V_{H} domains of a single arm of an antibody, (v) a domain antibody (dAb) fragment, which comprises a single variable domain; (vi) an isolated complementarity determining region (CDR); (vii) a single chain Fᵥ Fragment (scFᵥ); (viii) a diabody, which is a bivalent, bispecific antibody in which V_{H} and V_{L} domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem Fᵥ segments (V_{H}—C_{H}1-V_{H}-C_{H}1) which, together with complementarity light chain polypeptides, form a pair of antigen binding regions; (X) Dual-Variable Domain Immunoglobulin (xI) other non-full length portions of immunoglobulin heavy and/or light chains, or mutants, variants, or derivatives thereof, alone or in any combination.

The term “modified antibody format”, as used herein, encompasses antibody-drug-conjugates, Polyalkylene oxide-modified scFv, Monobodies, Diabodies, Camelid Antibodies, Domain Antibodies, bi- or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerised constructs comprising CH3+VL+VH, and the like. The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

Another preferred embodiment is an Immunoligand comprising at least one antibody or antibody fragment with binding capacity to MN as set forth in the above disclosure.

An “Immunoligand-Drug Conjugate” (IDC), as used herein, relates to a molecule that comprises a binding moiety of a humanized anti-MN antibody or antibody-based binding protein as disclosed herein, coupled to a small molecular weight active pharmaceutical ingredient (API), including, but not limited to a toxin (including e.g., but not limited to, tubulin inhibitors, actin binders, RNA polymerase inhibitors, DNA-intercalating and modifying/damaging drugs), a kinase inhibitor, or any API that interferes with a particular cellular pathway that is essential for the survival of a cell and/or essential for a particular physiologic cellular pathway.

Another preferred embodiment is an Immunoligand-Drug Conjugate as disclosed above comprising covalent a linker between an Immunoligand and preferably a small molecular weight active pharmaceutical ingredient (API).

In another preferred embodiment, said linker is at least one selected from the group consisting of
an oligopeptide linker, optionally comprising cleavable spacers, that may be cleaved by changes in pH, redox potential and or specific intracellular enzymes and/or
a maleimide linker, optionally comprising cleavable spacers, that may be cleaved by changes in pH, redox potential and or specific intracellular enzymes

In a preferred embodiment, the linker comprises, or consists of, at least one selected from the group consisting of: an oligopeptide linker (including cleavable and non-cleavable oligopeptide linkers), a hydrazine linker, a thiourea linker, a self-immolative linker, a succinimidyl trans-4-(maleimidylmethyl)cyclohexane-1-carboxylate (SMCC) linker, a maleimide linker, a disulfide linker, a thioether linker, and/or a maleimide linker.

The skilled person understands that further linkers may be suitable. Such linkers may be non-cleavable or may be cleaved by changes in pH, redox potential or specific intracellular or tumor tissue associated enzymes. Cleavable oligopeptide linkers include protease- or matrix metalloprotease-cleavable linkers. It is understood that the linker may comprise combinations of the above. For example, the linker may be a valine-citruline PAB linker.

In still another preferred embodiment, the linker comprises an oligopeptide of the sequence LPXTGₙ, with n being an integer between ≥1 and ≤20, and X being any amino acid.

In another preferred embodiment, the linker is conjugated to the C-terminus of at least one subdomain of the Immunoligand.

In another preferred embodiment, prior to conjugation,
the immunoligand bears a sortase recognition tag used or conjugated to the C-terminus of at least one subdomain thereof, and
the toxin comprises a short glycine stretch with a length of 1-20 glycine residues, preferably with a length of 3 to 5 amino acids.

In a preferred embodiment, the linker comprises an oligopeptide that is recognized by sortase enzymes, including but not limited to amino acid sequences selected from LPXSGₙ, LPXAGₙ, LPXTGₙ, LAXTGₙ, LAETGₙ, LPXTAₙ or NPQTGₙ with n being an integer between ≥1 and ≤21, and X being any amino acid.

In still another preferred embodiment, the linker comprises an oligopeptide of the sequence LPXTGₙ with n being an integer between ≥1 and ≤20, and X being any naturally occurring amino acid.

In another preferred embodiment, the linker is conjugated to the C-terminus of at least one subdomain of the Immunoligand.

In another preferred embodiment, prior to conjugation,
the immunoligand bears a sortase recognition tag used or conjugated to the C-terminus of at least one subdomain thereof, and
the toxin comprises a short glycine stretch with a length of 1-20 glycine residues, preferably with a length of 3 to 5 amino acids.

Preferably, the sortase recognition tag is:
LPXTG or LPXAG, which is recognized by Staphylococcus aureus sortase A;
LPXSG, which is recognized by Staphylococcus aureus sortase A or an engineered sortase A 4S-9 from Staphylococcus aureus;
LAXTG, and particularly LAETG, which is recognized by engineered sortase A 2A-9 from Staphylococcus aureus;
LPXTA, which is recognized by Streptococcus pyogenes sortase A; or
NPQTN, which is recognized by Staphylococcus aureus sortase B.

The following table shows the recognition tags and the peptides derived therefrom to be part of the linker (with n being an integer between ≥1 and ≤21, and X being any amino acid):

| | “naked” | peptide that |
|---|---|---|
| | recogni- | eventually |
| | tion | appears in |
| Sortase type | tag | the linker |
| Staphylococcus aureus | LPXTG | LPXTGₙ |
| sortase A | LPXAG | LPXAGₙ |
| Staphylococcus aureus | LPXSG | LPXSGₙ |
| sortase A or an engineered | | |
| sortase A 4S-9 from | | |
| Staphylococcus aureus | | |
| engineered sortase A 2A-9 | LAXTG | LAXTGₙ |
| from Staphylococcus | LAETG | LAETGₙ |
| aureus | | |
| Streptococcus pyogenes | LPXTA | LPXTAₙ |
| sortase A | | |
| Staphylococcus aureus | NPQTN, | NPQTGₙ |
| sortase B | | |

Engineered sortases, including A 2A-9 and A 4S-9 from Staphylococcus aureus, are described in Dorr B M et al., PNAS 2014; 111, 13343-8., and Chen et al., PNAS 2011; 108(28); 11399-11404.

As background and to exemplify the general concept of sortase transpeptidation, Sortase A, for example, uses an oligo-glycine-stretch as a nucleophile to catalyze a transpeptidation by which the terminal amino group of the oligo-glycine effects a nucleophilic attack on the peptide bond joining the last two C-terminal residues of the sortase tag. This results in breakage of that peptide bond and the formation of a new peptide bond between the C-terminally second-to-last residue of the sortase tag and the N-terminal glycine of the oligo-glycine peptide, i.e. resulting in a transpeptidation.

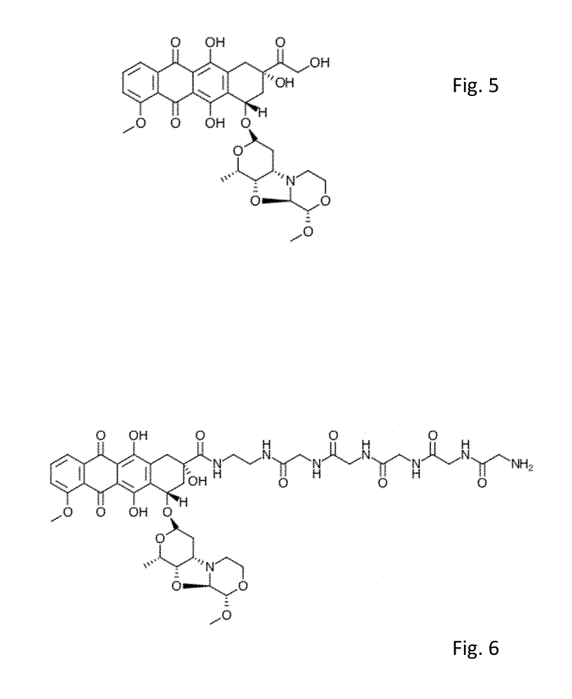

Prior to sortase conjugation, the sortase tag may, at its C-terminus, furthermore carry other tags, like His-tags, Myc-tags or Strep-tags (see FIG. 4a of WO2014/140317, the content of which is incorporated by reference herein). However, because the peptide bond between the 4th and 5th amino acid of the sortase tag is cleaved upon sortase A mediated conjugation, these additional tags do not appear in the conjugated product.

Sortase tag may, for example, be fused to a C-terminus of a binding protein, or to a domain or subunit thereof, by genetic fusion, and are co-expressed therewith. In another preferred embodiment, the sortase tag may directly be appended to the last naturally occurring C-terminal amino acid of the immunoglobulin light chains or heavy chains, which in case of the human immunoglobulin kappa light chain is the C-terminal cysteine residue, and which in the case of the human immunoglobulin IgG1 heavy chain may be the C-terminal lysine residue encoded by human Fcγ1 cDNA. However, another preferred embodiment is also to directly append the sortase tag to the second last C-terminal glycine residue encoded by human Fcγ1 cDNA, because usually terminal lysine residues of antibody heavy chains are clipped off by posttranslational modification in mammalian cells. Therefore, in more than 90% of the cases naturally occurring human IgG1 lacks the C-terminal lysine residues of the IgG1 heavy chains.

Therefore, one preferred embodiment of the invention is to omit the C-terminal lysine amino acid residues of human IgG1 heavy chain constant regions in expression constructs for sortase recognition-motif tagged Igγ1 heavy chains. Another preferred embodiment is to include the C-terminal lysine amino acid residues of human IgG1 heavy chain constant regions in expression constructs for sortase recognition-motif tagged Igγ1 heavy chains.

In another preferred embodiment the sortase tag may be appended to the C-terminus of a human immunoglobulin IgG1 heavy chain where the C-terminal lysine residue encoded by human Fcγ1 cDNA is replaced by an amino acid residue other than lysine to prevent unproductive reactions of sortase with the ε-amino group of said C-terminal lysine residue leading to inter-heavy chain crosslinking.

We have described previously that in some cases (e.g. at the C-terminus of the Ig kappa light chains, see: Beerli et al. (2015) PloS One 10, e131177) it is beneficial to add additional amino acids between the C-terminus of the binding protein and the sortase tag. This has been shown to improve sortase enzyme conjugation efficiencies of payloads to the binding protein. In the case of Ig kappa light chains, it was observed that by adding 5 amino acids between the last C-terminal cysteine amino acid of the Ig kappa light chain and the sortase pentapeptide motif improved the kinetic of conjugation, so that the C-termini of Ig kappa light chains and Ig heavy chains could be conjugated with similar kinetics (see: Beerli et al. (2015) PloS One 10, e131177). Therefore, it is another preferred embodiment that optionally ≥1 and ≤11 amino acids are added in between the last C-terminal amino acid of a binding protein or antibody subunit and the sortase tag.

Further, the immunoligand can bear, C-terminally of the sortase tag, other tags, like a His tag, a Myc tag, Strepll tag and/or a. TwinStrep tag. See WO2014140317 A2 for more details, the subject matter of which is incorporated by reference herein

In another preferred embodiment, the toxin is at least one selected from the group consisting of
maytansinoids,
auristatins,
anthracyclins, preferably PNU-derived anthracyclins
calcheamicins,
tubulysins
duocarmycins
radioisotopes
liposomes comprising a toxid paylooad,
protein toxins
taxanes, and/or
pyrrolbenzodiazepines.

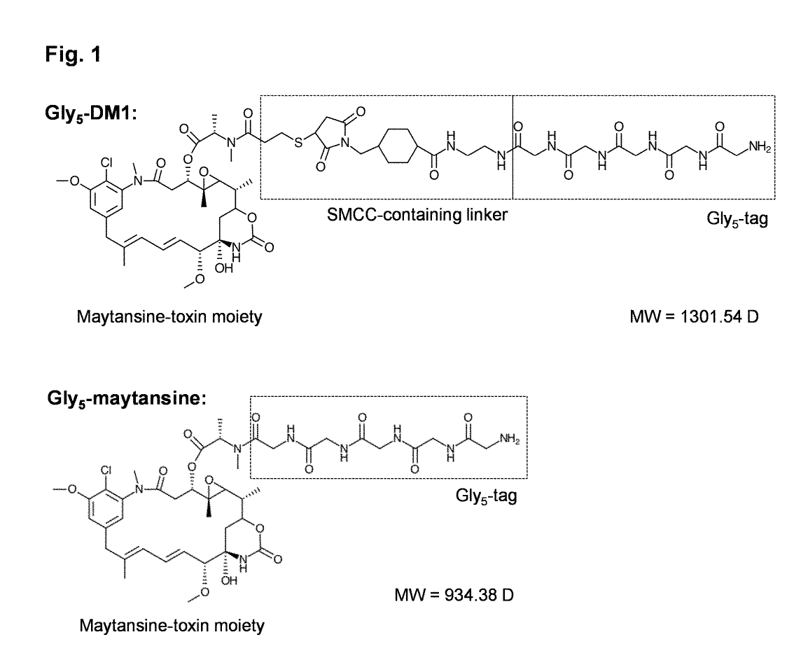

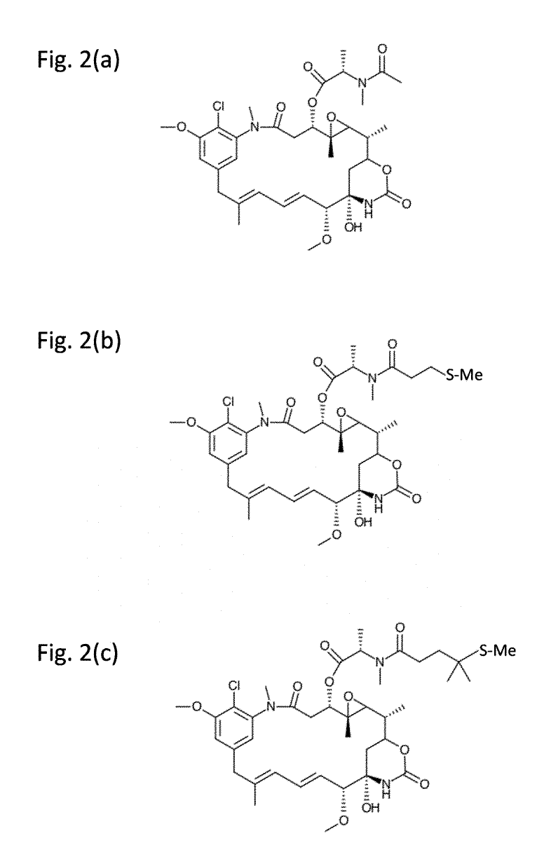

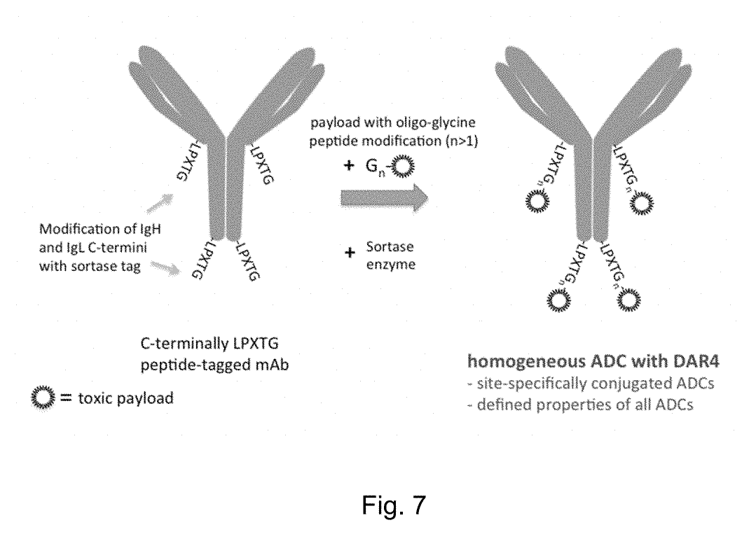

Examples for preferred maytansinoid toxins are shown in FIGS. 1 and 2. The anthracycline derivatives disclosed herein are also nicknamed as “PNU”, and are derivatives of PNU-159682, which is a metabolite of the anthracycline nemorubicin and has for the first time been disclosed by Quintierei et al. 2005. PNU-159682 is shown FIG. 5.

Immunoligand Drug Conjugates comprising anthracycline derivatives are disclosed in WO2016102697 and applications claiming the priority thereof, the content of which is incorporated by reference herein.

Preferably, the Immunoligand Drug Conjugates comprises two or more different toxins. In such way, the cell killing activity can be enhanced, e.g. by avoiding resistances against monotoxins, or by cooperative action of the two toxins.

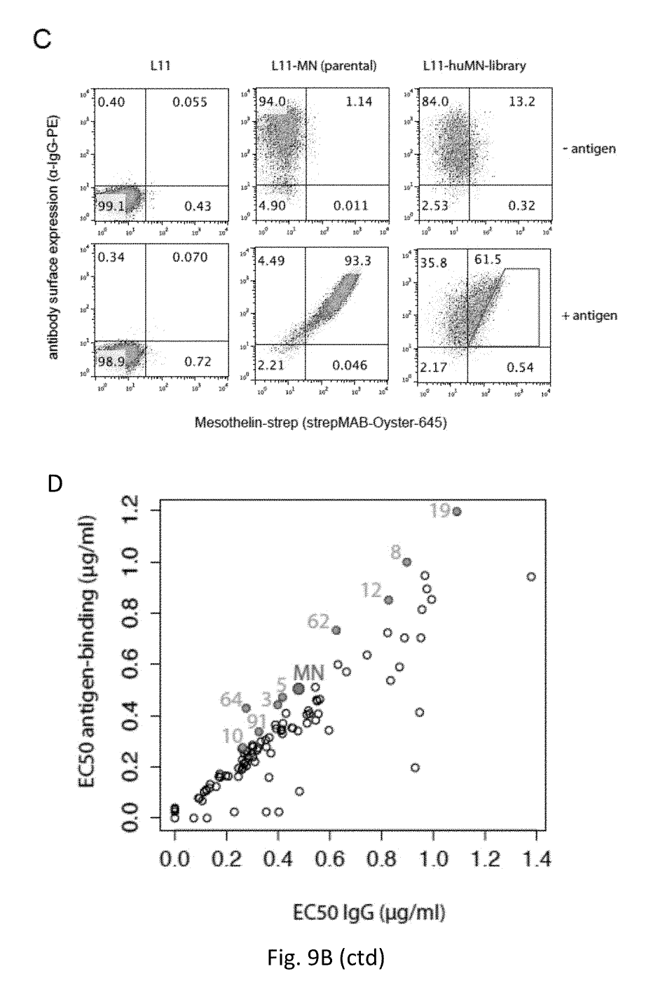

In another preferred embodiment, the Immunoligand-Drug Conjugate has a cell killing activity as set forth in FIG. 8.

In another preferred embodiment, the Immunoligand-Drug Conjugate is created by sortase-mediated conjugation of (i) an Immunoligand carrying one or more sortase recognition tags and (ii) one or more toxins carrying an oligoglycine tag.

According to another aspect of the invention, a method of producing an Immunoligand-Drug Conjugate according to any of the aforementioned disclosure is provided, which method comprises the following steps:
a) providing an Immunoligand according to the list set forth above, which Immunoligand carries a sortase recognition tag,
b) providing one or more toxins carrying an oligoglycine tag, and
c) conjugating the Immunoligand and the toxin by means of sortase-mediated conjugation.

The method of conjugating an Immunoligand to a payload by means of a sortase or a split intein is disclosed in full detail in WO2014140317 A2, the subject matter of which is incorporated by reference herein.

According to yet another embodiment, a MN specific chimeric antigen receptor (CAR) is provided, comprising
a) at least one antibody, antibody-based binding protein, modified antibody format or antibody derivative or fragment according to the above description, or
b) a bi- or multispecific antibody according to the above description,
   which is fused or conjugated to at least one transmembrane region and at least one intracellular domain.

Chimeric antigen receptors (CAR), sometimes also called artificial T cell receptors, are engineered receptors, which graft an arbitrary specificity onto an immune effector cell. Typically, these receptors are used to graft the specificity of a monoclonal antibody onto a T cell; with transfer of their coding sequence facilitated by retroviral vectors. The receptors are called chimeric because they are composed of parts from different sources.

CARs are potential candidates as a therapy for cancer, using a technique called adoptive cell transfer. T cells are removed from a patient and modified so that they express CARs specific to the patient's particular cancer, by specifically binding to a cancer-specific antigen, as is the case in ROR1. The T cells, which can then recognize and kill the cancer cells, are reintroduced into the patient.

The structure of the prototypic CAR is modular, designed to accommodate various functional domains and thereby to enable choice of specificity and controlled activation of T cells. In the context of the present invention, a CAR comprises an antibody-like binding domain derived from an antibody, antibody-based binding protein, modified antibody format, antibody derivative or fragment, which targets MN. Such entity can be, e.g., but is not limited to, a single chain variable fragment (scFv) that combines the specificity and binding residues of both the heavy and light chain variable regions of a monoclonal antibody in a single polypeptide chain, fused or conjugated to at least one transmembrane region and at least one intracellular domain.

Preferably, said transmembrane region comprises a CD8a transmembrane domain. Preferably, said CAR further comprises a hinge region disposed between the transmembrane domain and the antibody, antibody-based binding protein, modified antibody format retaining target binding capacity, or antibody derivative. Preferably, said intracellular domain comprises a T-cell receptor signaling domain. More preferably, said signaling domain comprises or is derived from a zeta chain of a CD3-zeta chain. Preferably, said intracellular domain further comprises one or more intracellular signaling domain of a T cell costimulatory molecule.

A preferred intracellular signaling domain of a T cell costimulatory molecule is selected from the group consisting of 4-1BB, CD-28, OX40 and/or CD278/ICOS. Combination of two or more of these domains are specifically preferred.

According to another embodiment of the invention, a cell comprising such chimeric antigen receptor is provided.

Said cell is preferably an engineered T cell, also called “CAR T cell”. CAR T cells are genetically engineered T cells armed with CARs whose extracellular recognition unit is comprised of an antibody-derived recognition domain and whose intracellular region is derived from lymphocyte stimulating moiety(ies). By arming T cells with such chimeric receptors, the engineered cell is redirected with a predefined specificity to any desired target antigen, in a non-HLA restricted manner CAR constructs are introduced ex vivo into T cells from peripheral lymphocytes of a given patient using retroviral or lentiviral vectors or transposable elements. Following infusion of the resulting CAR-engineered T cells back into the patient, they traffick, reach their target site, and upon interaction with their target cell or tissue, they undergo activation and perform their predefined effector function. Therapeutic targets for the CAR approach include cancer and HIV-infected cells, or autoimmune effector cells. Alternatively, said cell is preferably an engineered natural killer cell (NK cell).

Another aspect of the invention is the use of the antibody-based binding protein, modified antibody format retaining target binding capacity, antibody derivative or fragment of any of claims according to the above description, the bi- or multispecific antibody according to the above description, the Immunoligand-Drug Conjugate according to the above description, or the CAR or cell according to the above description, for the treatment of a patient that is
suffering from,
at risk of developing, and/or
being diagnosed for
   a neoplastic disease.

In a preferred embodiment, the neoplastic disease is at least one selected from the group of solid cancers, preferably
Pancreatic adenocarcinoma,
Mesothelioma
Lung cancer
Ovarian cancer
Breast cancer, preferably triple negative breast cancer, and/or
Cholangiocarcinoma

According to a further aspect of the invention, a pharmaceutical composition is provided, which comprises the antibody or antibody-based binding protein, modified antibody format retaining target binding capacity, antibody derivative or fragment according to the above description, the bi- or multispecific antibody according to the above description, the Immunoligand-Drug Conjugate according to the above description, or the CAR or cell according to the above description, together with one or more pharmaceutically acceptable ingredients.

According to a further aspect of the invention, a method of killing or inhibiting the growth of a cell expressing MN in vitro or in a patient is provided, which method comprises administering to the cell a pharmaceutically effective amount or dosis of (i) the antibody or antibody-based binding protein, modified antibody format retaining target binding capacity, antibody derivative or fragment according to the above description, the bi- or multispecific antibody according to the above description, the Immunoligand-Drug Conjugate according to the above description, or the CAR or cell according to the above description, or (ii) of a pharmaceutical composition according to the above description

Preferably the cell expressing MN is a cancer cell, preferably, Pancreatic adenocarcinoma, Mesothelioma, and/or Lung cancer.

Further preferably, the MN is human MN.

### Experiments and Figures

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Materials and Methods

The following section describes an experimental campaign where anti-Mesothelin and anti-ROR1 antibodies were created. Nonetheless, most of the experimental details apply at least non-exclusively to anti-ROR1 antibodies.

1. Antibody Production

Parental anti-ROR1 mouse mAb 2A2[1] and rabbit mAb R11[2] and anti-Mesothelin mouse mAb MN[3,4] were produced as chimeric full-length IgG1 antibodies with human constant regions as follows. Variable region coding regions were produced by total gene synthesis (GenScript, Piscataway, USA) using MNFGLRLIFLVLTLKGVQC as leader sequence, assembled with human IgH-γ 1 and IgL-κ constant regions in the expression vector pCB14b, and expressed in 293T cells. pCB14b is a derivative of the episomal mammalian expression vector pCEP4 (Invitrogen), carrying the EBV replication origin and encoding the EBV nuclear antigen (EBNA-1) to permit extrachromosomal replication, and contains a puromycin selection marker in place of the original hygromycin B resistance gene.

2. Expression and Purification of Antigens

StrepII-tagged ROR1-extracellular domain was produced as follows: the nucleotide sequence encoding the expracellular domain of human ROR1 (NP_005003) was N-terminally fused to a signal sequence (MNFGLRLIFLVLTLKGVQC) and C-terminally fused with a sequence encoding a strepII-tag (GWSHPQFEK). StrepII-tagged Mesothelin was produced as follows: the nucleotide sequence encoding the human mesothelin isoform 2 (NP_037536) was N-terminally fused to a signal sequence (MNFGLRLIFLVLTLKGVQC) and C-terminally fused with a sequence encoding a strepII-tag (GWSHPQFEK). The entire nucleotide sequences with flanking 5′NotI and 3′HindIII sites were produced by total gene synthesis (GenScript, Piscataway, USA), assembled in the proprietary mammalian expression vector pEvi5 by Evitria (Schlieren, Switzerland) and verified by DNA sequencing. Expression of the proteins was performed in suspension-adapted CHO K1 cells by Evitria (Schlieren, Switzerland). Supernatants from pools of transfected CHO K1 cells were harvested by centrifugation and sterile filtered (0.2 μm) before FPLC-based affinity purification using StrepTactin columns (IBA GmbH, Goettingen, Germany).

3. Construction of TranspoMab Vectors

All DNA syntheses to generate plasmids were performed by GenScript (Piscataway, USA). The amino-acid sequence of hyperactive PiggyBac transposase (hyPB) according to [5] and was codon-optimized for murine expression, synthesized and cloned into pcDNA3.1. Transposable antibody expression constructs (pPB) were assembled from modular parts with flanking restriction sites that were synthesized or derived from sequence-verified commercially available vectors, and are described in detail in Patent WO2014013026A1 Antibody ORFs were assembled in transposable vectors as follows: variable regions along with the leader sequence MNFGLRLIFLVLTLKGVQC were introduced using 5′NotI/3′NheI (IgHV) or 5′NotI/3′BsiWI (IgkappaV) restriction sites, in-frame using 5′NheI/3′BstBI (IgHC-gamma 1) or 5′BsiWI/3′BstBI (IgKC) restriction sites.

4. Library Construction

Variable regions were synthesized by Gen9, Inc. (Cambridge, USA), pooled to equimolar amounts and amplified by PCR using forward primer univ-Not1-SP-F

(GAGGAGGCGGCCGCCATGAACTTTGGG)

and reverse primers huCG1-B

(AAGACCGATGGGCCCTTGGTG)

for IgHV and huCK-B

(GAAGACAGATGGTGCAGCCAC).

Cycling conditions were: 98° C./60 sec->25× (98° C./45 sec, 56° C./45 sec, 72° C./60 sec)->72° C., 5 min->hold@ 4° C. Amplified fragments were column-purified, digested using NotI/NheI (IgHV) or NotI/BsiWI (IgkappaV) and cloned into transposable vectors by 2-way (HC constructs) or 3-way cloning (LC constructs): Vector fragments were prepared by digestion with NotI/NheI (pPB-Hygro-HCg1-gen) or by digestion with NotI/BstBI as well as with BsiWI/BstBI (pPB-Puro-LC). Library ligations were transformed into Neb5-alpha electrocompetent cells (Neb, Ipswich, USA), pre-cultured for 1 hour, amplified in selective LB-media containing 0.1 mg/ml ampicillin overnight and plasmid DNA was isolated using NucleoBond Xtra Maxi Plus kit (Macherey&Nagel, Dueren, Germany) Library sizes were determined by plating out serial dilutions of the pre-culture onto selective agar plates (titration plates) and obtained clone numbers were backcalculated to obtain library sizes. At least 12 clones from titration plates were analyzed by restriction digest and sequencing of variable regions using primer pPBseq13 (GGCCAGCTT GGCACTTGATG).

5. Cells

L11 cells represent an in-house generated subclone of the Abelson murine leukemia virus (A-MuLV) transformed pre-B cell line 63-12 isolated from RAG-2 deficient mice [6] and were cultured in SF-IMDM media supplemented with 2% fetal calf serum, 2 mM L-Glutamine, 100 IU Penicillin, 0.1 mg/ml Streptomycin (Amimed, BioConcept Ltd., Allschwil, Switzerland) and 50 μM b-mercaptoethanol (Amresco, Solon, USA) in screwcap bottles (Sarstedt, Nümbrecht, Germany) at 37° under 7.5% CO₂.

EMT6 cells (ATCC, CRL-2755), a kind gift of Prof. A. Zippelius (University of Basel) and 293T cells (ATCC, CRL-3216) were both grown in DMEM supplemented with 10% FCS, 2 mM L-Glutamine, 100 IU Penicillin, 0.1 mg/ml Streptomycin and 0.25 μg/ml Fungizone (Amimed) at 37° under 5% CO₂.

6. Transposition and Selection of L11 Cells

One day before electroporation, L11 cells were seeded at a density of 0.2E+6 cells/ml to obtain log-phase growing cells the next day. The entire procedure of electroporation was performed at room temperature. Cells were harvested by centrifugation at 1200 rpm for 6 min and resuspended in plain RPMI medium to a concentration of 8E+7 cells/ml. Per cuvette, 25 μg of total DNA was diluted in 400 μl RPMI (using HC/LC/tranposase weight ratios as shown in Figure S2B) and 400 μl cell suspension was combined with diluted DNA and transferred to a 0.4 cm gap gene pulser cuvette (BioRad, Hercules, USA). Electroporation was done with a BioRad GenePulser II equipped with capacitance extender set to 300V and 950 μF. After incubation for 5-10 min in cuvettes in order to allow pores to close, cells were washed once in complete SF-IMDM growth medium, resuspended and seeded into T175 tissue culture flasks at a total volume of 64 ml of complete growth medium. For selection, 1 μg/ml Puromycin and 800 μg/ml Hygromycin (0240.4 and CP12.2, respectively; Carl Roth, Karlsruhe, Germany) were added simultaneously and selection was allowed to proceed for 4-5 days without exchange of medium or subculturing, until selection was complete.

7. Staining and Sorting of Cellular Libraries

Cells were stained on ice in FACS-buffer (PBS supplemented with 2% FCS) at a concentration of 1E+7 cells/ml. Washes were performed by pelleting cells by centrifugation at 1300 rpm for 3 min, resuspension in FACS-buffer using a 5× volume of staining reactions, pelleting again and resuspension in FACS buffer using 1× volume of staining reaction.

For analysis of surface-antibody expression, cells were stained using 1:200 diluted Ig-kappaLC-APC labeled antibody (MH10515, Life Technologies, Carlsbad, USA) for 30 minutes. Cells were washed once and analysed by flow cytometry on a FACSCalibur instrument (Becton-Dickinson, Franklin Lakes, USA).

For staining of cellular libraries, previously determined limiting concentrations of antigen (0.12 μg/ml Mesothelin-strep; 0.25 μg/ml ROR1-strep) and 1:250 diluted anti-Human-IgG (Fc gamma-specific) PE (ebioscience 12-4998-82) were added and cells were incubated on ice in the dark for 30 minutes. After washing cells once, 1:500 diluted Strep-mAb classic Oyster 645 (2-1555-050 iba, Goettingen, Germany) was used to detect strep-tagged antigen bound to cells for 30 minutes. After a final wash, cells were filtered using cell strainer cap FACS tubes (BD Falcon). Sorting was performed on a FACSAriaII instrument (Becton-Dickinson, Franklin Lakes, USA).

8. ELISA

Antigen-binding analysis by ELISA was performed by coating of Nunc-Immuno MaxiSorp 96-well plates (Thermo Scientific, Waltham, USA) with antigen diluted in coating buffer (100 mM bicarbonate/carbonate buffer) over night at 4° C. For sandwich ELISA, plates were coated with 2 μg/ml AffiniPure F(ab′)2 fragment donkey anti-human IgG (Jackson Immunoresearch, West Grove, USA) diluted in coating buffer over night at 4° C. After coating, plates were washed twice with PBS/0.05% Tween-20 (PBS-T), blocked with PBS-T containing 3% bovine serum albumin (BSA) (Carl Roth, Karlsruhe, Germany) for 1 hour at 37° C. and washed again 5 times with PBS/T. L11 clone supernatants were pre-diluted 3-fold, while supernatants from 293T cells were pre-diluted 50-fold. Parental mAbs diluted to 0.5 μg/ml were used to generate standard curves. 3.5-fold serial dilutions of samples were added and plates were incubated for 1 hour at 37° C. After 5 washes with PBS/T, HRPO-conjugated F(ab)2 anti-human FC-gamma (Jackson Immunoresearch, West Grove, USA) was added at 10′000-fold dilution in PBS/T containing 1% BSA, and plates were incubated for 1 hour at 37° C. Finally, plates were washed 5 times with PBS/T and 50 μl of Sigmafast OPD Peroxidase substrate (Sigma-Aldrich, St. Louis, USA) were added. Reactions were stopped by adding 50 μl of 2M H₂SO₄. Absorption was measured at 490 nm. OD50 values of standards with known concentrations and samples determined by 4-point curve fitting models were used to calculate EC50.

9. SPR

Affinities were determined using a Biacore T200 instrument (GE Healthcare, Buckinghamshire, UK) and data was evaluated using Biacore Evaluation T200 V2.0 software. To capture mAbs, goat a-human Fc-gamma-specific IgG (Jackson ImmunoResearch, #109-005-098) was covalently immobilized on a CMS chip (GE Healthcare, # BR-1005-30).

For determination of huMN affinities, 293T supernatants containing mAbs were diluted to 10 μg/ml IgG with running buffer (HBS-EP+pH 7.4 (10 mM HEPES, 150 mM NaCl, 3 mM EDTA, 0.05% Tween 20) and captured for 60 s with a flow of 10 μl/min Mesothelin-strep was diluted in running buffer using 2-fold serial dilutions ranging from 10 nM to 1.25 nM. Association was measured at a flow of 30 μl/min for 120 s, and dissociation was followed for 1000 s. Capture levels ranged from 90.0 RU to 493.6 RU.

For measurement of L11 hu2A2 and huR11 clone supernatants, antibodies were diluted to 0.3 μg/ml with complete SF-IMDM cell culture medium and captured for 120 s with a flow of 10 μl/min ROR1-strep was diluted to 40 nM in running buffer. Association was measured at a flow rate of 30 μl/min for 120 s, and dissociation was followed for 200 s. Curves were fitted using 30 s dissociation due to upper plateau formation at later timepoints. Capture levels ranged from 29.1 RU to 57.7 RU.

For determination of hu2A2 and huR11 affinities, purified mAbs were diluted to 0.3 μg/ml with running buffer and captured for 120 s with a flow of 10 μl/min ROR1-strep was diluted in running buffer using 2-fold serial dilutions ranging from 20 nM to 2.5 nM. Association was measured at a flow of 30 μl/min for 120 s, and dissociation was followed for 200 s. Curves were fitted using 30 s dissociation due to upper plateau formation at later timepoints. Capture levels ranged from 29.1 RU to 57.7 RU.

All measurements were performed at 25° C. All curves were fitted using a 1:1 binding model with RI=0. Regeneration was done for 90 s using 100 mM H₃PO₄ at a flow of 30 μl/min.

10. Sequence Recovery

RNA was extracted from ˜2E+6 cells grown in 24-well plates using Tri-Reagent (Sigma-Aldrich, St. Louis, USA) and reverse transcribed with ProtoScriptII Reverse transcriptase (Neb, Ipswich, USA) using random nonamers, according to manufacturer's instructions. Variable regions were amplified by PCR using Q5 DNA polymerase (Neb, Ipswich, USA) by means of forward primer EF1allotI_F (CCATTTCAGGTGTCGTGAGC) and reverse primers CG-revseq-1 (GTTCGG GGAAGTAGTCCTTG) for VH and Intron-rev-1 (GTGGATGTCAGTAAGACCAATAGGTGCC) for VL. Cycling conditions were 98° C./30 sec->30× (98° C./20 sec, 58° C./20 sec, 72° C./45 sec)->72° C./5 min->hold@ 4° C. PCR products were purified by column purification (Macherey&Nagel, Dueren, Germany), digested and again purified by agarose-gel purification. Recovered variable regions were assembled in pCB14b, along with human Ig-gamma1 or Ig-kappa constant regions by 2- or 3-way cloning. Variable regions from several bacterial clones were sequenced by Sanger sequencing at Microsynth AG (Balgach, Switzerland) using primer CMVseq2 (GCAGTGTAGTCTGAGCAGTAC) and were aligned to library sequences using Geneious Software (Biomatters, New Zealand).

11. Expression of Antibodies

Expression of antibodies was achieved by transfecting pCB14b-based expression constructs into HEK-293T cells and harvesting cell supernatants.

For transient antibody expression, cells were transfected in 6-well-plates using Lipofectamine LTX plus (LifeTechnologies, Carlsbad, USA). Per well, 2.5 μg of total DNA was transfected, and fresh growth medium was added the next day and conditioned for 4 days. Supernatants were sterile-filtered and stored at −20° C. until analysis.

For large-scale antibody expression, cells were transfected in 10 cm dishes using Lipofectamine LTX plus, enriched by selection with 2 μg/ml Puromycin (0240.4, Carl Roth, Karlsruhe, Germany), expanded to 14 cm dishes coated with poly-L lysine and maintained in DMEM/F12 serum-free medium (Gibco) containing 161 μg/ml N-Acetyl-L-Cysteine, 10 mg/ml L-Glutathione and 1 μg/ml Puromycin. Supernatants containing the respective antibodies were harvested twice a week, sterile-filtered and stored at 4° C. until purification. Purification by FPLC was done on an Akta purifier instrument (GE Lifesciences). After passing supernatants over Amsphere protein A columns (JWT203CE, JSR Micro, Sunnyvale, USA), followed by washing with PBS, antibodies were eluted with 0.1M Glycine pH 2.5 and immediately neutralized with 1M Tris pH 8.0. Buffer exchange with PBS was performed using Amicon Ultra-4 Centrifugal Filters (Merck Millipore).

12. Secondary ADC Assay

Secondary-ADC-mediated cytotoxicity of antibody-containing L11 clone supernatants was investigated using clone #6 of EMT6-Meso murine breast cancer cells overexpressing Mesothelin. Cells were plated on 96-well plates in 75 μl growth medium at a density of 10,000 cells per well and grown at 37° C. in a humidified incubator in a 5% CO2 atmosphere. After one day, growth medium was replaced with 50 μl L11 supernantants that were serially diluted 3.5-fold with complete growth medium. After incubation for 30 min under growth conditions, 50 μl of 3.5-fold serial dilutions of secondary ADC was added (aHFc-CL-MMAE, MORADEC AH-102PN). Each dilution was done in duplicate. After incubation for 3 days under growth conditions, plates were removed from the incubator and equilibrated to room temperature. After approximately 30 minutes, 50 μl CellTiter—Glo Luminescent Solution (Promega, Cat.No G7570) was added to each well and, after shaking the plates at 650 rpm for 5 min, followed by a 10 min incubation without shaking, luminescence was measured on a Tecan Infinity F200 with an integration time of 1 second per well.

### Results

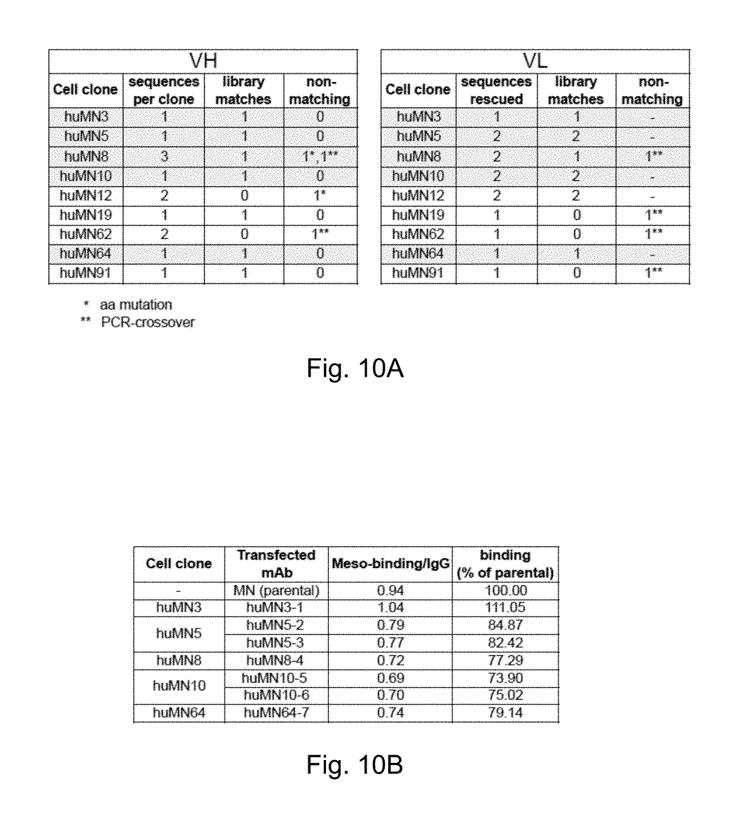

To generate a cellular library, one-step electroporation of HC and LC libraries along with transposase expression plasmid into L11 cells was performed, followed by selection in Hygromycin B and Puromycin one day after electroporation. We determined transposition efficiency by culturing a small part of the library without antibiotics for 3 days and analyzed antibody surface expression by flow cytometry (FIG. 9B, A). Consistent with previous results, around 7.5% cells expressed surface antibody when analyzed by flow cytometry. Thus, considering the small theoretical size of the mini-library (47×47=2,209 variants) and the number of cells electroporated (2.4E+6), we estimate that an approximately 1000-fold overrepresentation of the cDNA library was achieved. Antibiotic selection of the cellular library was complete after 4 days and highly efficient, as judged by surface antibody staining analyzed by flow cytometry (FIG. 9B, B). After subculturing of cells in non-selective media for one day in order to let cells recover from antibiotic selection, we proceeded to staining of the library for antigen binding (FIG. 9B, C). Flow cytometry analysis of the library demonstrated that a large portion of the cellular library was able to bind soluble antigen as expected, although the majority of cells appeared to display weaker binding compared to cells expressing the parental antibody. Based on these observations we directly proceeded to stringent sorting of single cells into 96-well plates. After 2 weeks, single cells had expanded and supernatants of 96 clones were harvested and analysed by ELISA (FIG. 9B, D). The majority of cell-clone derived supernatants showed antigen binding similar to that of the parental mAb when normalized to IgG secretion levels, while only a few showed little or no antigen binding and/or were devoid of IgG expression, demonstrating that cell sorting based on antigen-binding and surface-expression was highly efficient.

### Sequence Recovery, Validation and Affinities of Top Mesothelin Binders

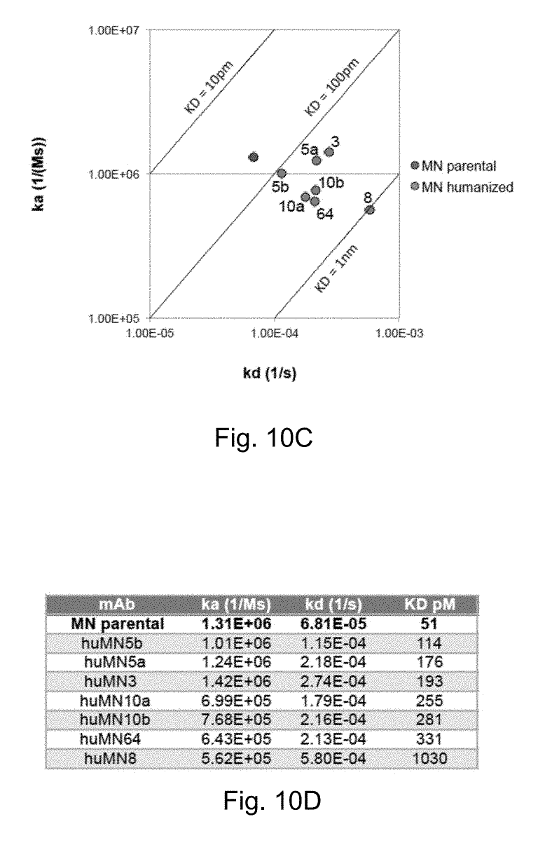

After direct evaluation of cell clone supernatants, we chose to recover the antibody variable region sequences of those nine cell clones that showed the best binding activity in ELISA (FIG. 9B, D). In order to do so, RNA was isolated and RT-PCR was performed using primers flanking HC or LC variable regions. Amplified VH and VL products were combined with human HC and LC constant regions by cloning into the episomal expression vector pCB14b. Sequencing of several minipreps from each cell clone followed by alignment of rescued sequences with library candidate sequences showed that several of the cell clones expressed more than one HC and/or LC, in line with our previous observations (FIG. 10A). Five out of the nine cell clones analysed contained HC/LC pairs that matched sequences of the designed candidate HC and LC libraries, whereas four cell clones contained at least one chain where none of the sequences matched library design. In the majority of cases these represented PCR-crossovers that presumably occurred between highly similar sequences during amplification of pooled library fragments, in line with our observation that crossed-over sequences were present in the cDNA library (data not shown). Clones containing these types of sequences were not pursued further.

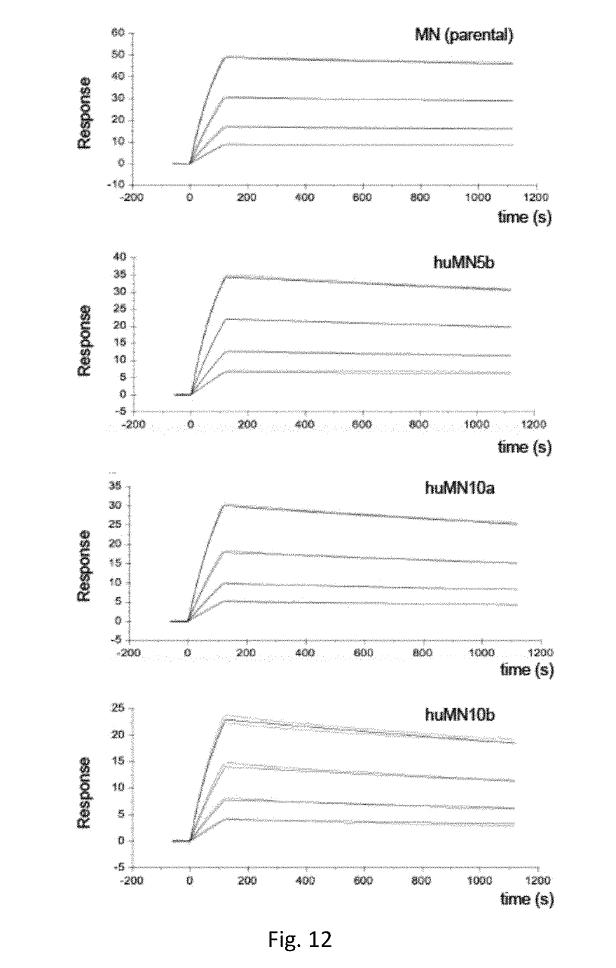

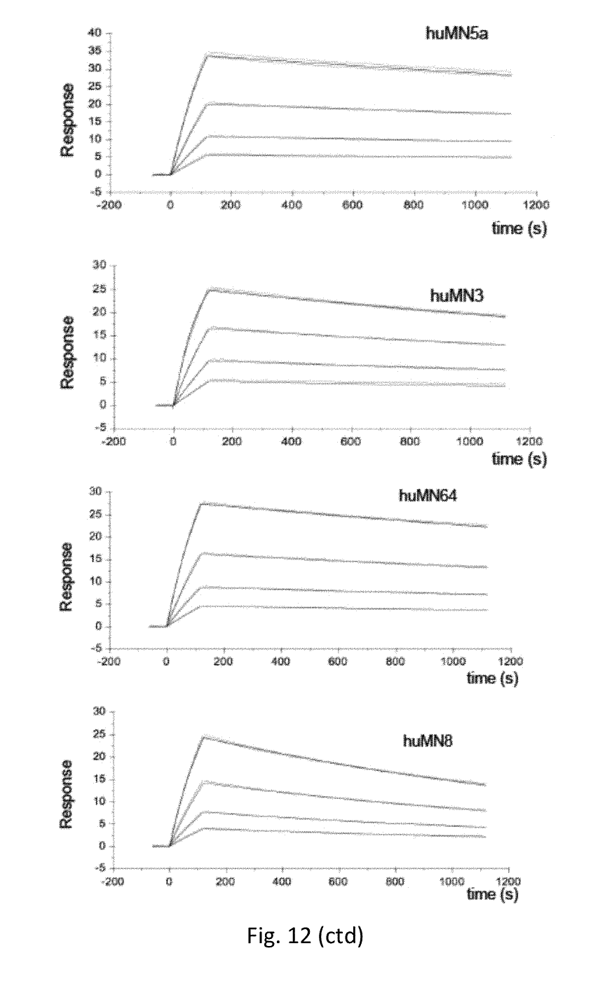

To verify antigen-binding of individual HC/LC pairs recovered from each cell clone, we transiently transfected 293T cells with the respective combinations of HC/LC constructs generated during sequence recovery, along with the parental chimeric antibody as a control. Supernatants were then analyzed for antigen-binding and IgG-titer by ELISA. This analysis demonstrated that all of the recovered sequences were indeed coding for functional Mesothelin-binders (FIG. 5B). To determine individual affinities of the entire set of ELISA-validated mAbs we analyzed the same supernatants by surface plasmon resonance (SPR) (FIGS. 10C and D, see FIG. 12 for response-curves). The best binder showed no more than a two-fold lower affinity (KD=114 pm) compared to the parental mAb MN (KD=51 pM), and the remaining clones showed affinities of between 176 and 1030 pM. Analysis of the degree of humanization among these clones was also performed. To do so, we determined the similarity of each chain's framework regions to those of the human germline sequence that was most closely related to the entire variable region sequence of the humanized mAbs (FIG. 13). Significantly, the clone with the lowest affinity in this set contained both HC and LC frameworks that were 99% identical to frameworks of the closest human germline sequence, while higher affinity clones deviated more strongly from the most closely related germline sequence. Overall, the average grade of humanization of the library and isolated mAbs compared well to humanized antibodies that have been clinically approved (FIG. 13), thus validating the humanization strategy. Collectively, the results obtained demonstrate that cellular antibody libraries can be easily generated by transposition, and high-affinity antibodies can be isolated in a straightforward fashion by screening of cell clone supernatants directly after enrichment for antigen binders by FACS.

### Functional Evaluation of Clone Supernatants

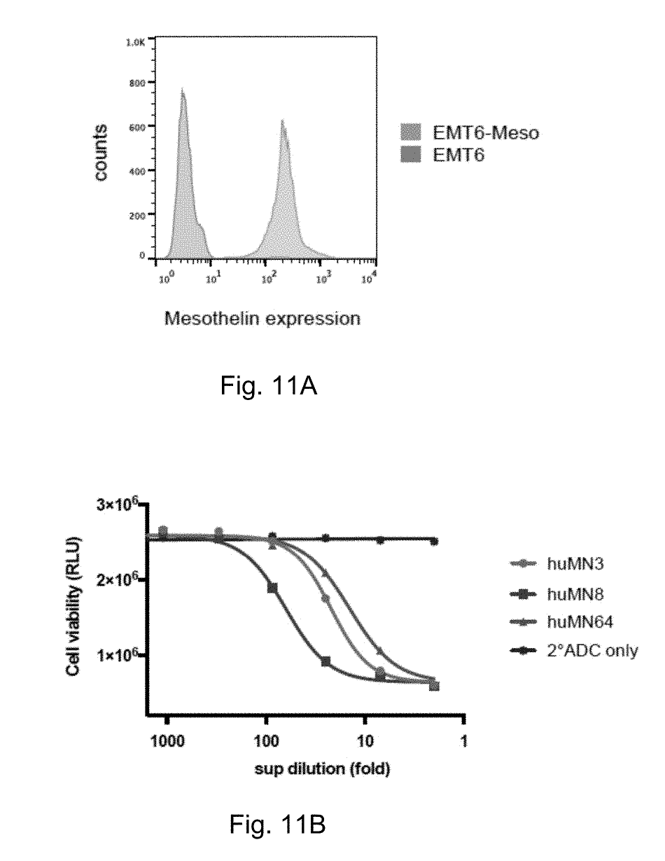

After having shown that antibody-containing supernatants from sorted L11 cell clones can directly be used for affinity measurements, we next wished to investigate whether other functional properties, such as suitability of mAbs as antibody drug conjugates (ADC), can directly be evaluated as well. The seamless integration of antibody discovery and evaluation of ADC-dependent in vitro cell killing activity would greatly facilitate ADC discovery, which typically requires the screening of large numbers of clones until a suitable mAb is identified. Thus, we chose to test the antibody-containing supernatants generated during our MN humanization screen directly in secondary ADC cell killing assays on EMT6-Meso cells, a subclone of mouse EMT6 breast cancer cells overexpressing human mesothelin (FIG. 11A). For this, EMT6-Meso cells were plated in 96-well format and exposed to serial dilutions of supernatants. After a brief incubation, a secondary ADC reagent was added consisting of a polyclonal anti-human Fc antibody conjugated to monomethyl auristatin E (MMAE) via a cleavable linker. While incubation with secondary ADC alone did not lead to cell death even when used at the highest concentration, combined incubation with antibody-containing supernatants resulted in dramatically reduced cell viability, indicating antigen-specific cell killing via mAb-binding and internalization of mAb-ADC complexes (FIG. 11B). These results demonstrate that TranspoMab is not only a powerful antibody discovery and engineering platform, but also allows for seamless integration of functional screening without the need for antibody re-formatting or re-cloning.

Table 1. Affinities of parental mouse mAb MN and humanized versions thereof. To evaluate the binding strength (affinity) of the generated humanized monoclonal antibodies (mAbs), and to compare affinites with parental mAbs, surface plasmon resonance (SPR) measurements were conducted, a biophysical method to accurately determine affinity. SPR was performed using a Biacore T200 instrument (GE Healthcare, Buckinghamshire, UK) and data was evaluated using Biacore Evaluation T200 V2.0 software. To capture mAbs, goat a-human Fc-gamma-specific IgG (Jackson ImmunoResearch, #109-005-098) was covalently immobilized on a CMS chip (GE Healthcare, # BR-1005-30).

For determination of affinities, mAbs were diluted in running buffer HBS-EP+pH 7.4 (10 mM HEPES, 150 mM NaCl, 3 mM EDTA, 0.05% Tween 20) to 10 μg/ml with running buffer and captured for up to 60 s with a flow of 10 μl/min. Mesothelin-strep was diluted in running buffer using 2-fold serial dilutions ranging from 10 nM to 1.25 nM. Association was measured at a flow of 30 μl/min for 120 s, and dissociation was followed for 1000 s. All measurements were performed at 25° C. All curves were fitted using a 1:1 binding model with RI=0. Regeneration was done for 90 s using 100 mM H3PO4 at a flow of 30 μl/min.

FIG. 1: Chemical structures of the Gly₅ modified toxins used for SMAC-Technology™ immunoligand conjugation.

In the upper part of FIG. 1 the maytansinoid is DM1 ([N²′-deacetyl-N²′-(3-mercapto-1-oxopropyl)-maytansine], containing the so-called SMCC linker to which the oligo-glycine peptide (Glyₙ) was coupled, in order to allow conjugation by SMAC-Technology™, but to provide the same chemical structure of the DM1 payload in SMAC-Technology™ conjugated HER-2 ADCs as in chemically conjugated trastuzumab-DM1. However, this SMCC linker is only an optional component for the SMAC-Technology™ conjugated immunoligand toxin conjugates, and of no importance for the conjugation of the payload.

Instead of DM1, other optional linker structures, like the SPDB linker of the maytansinoid payload DM4 ([N20-deacetyl-N20-(4-mercapto-4-methyl-1-oxo-pentyl)-maytansine] may optionally be included, see FIG. 2.

In the lower part of FIG. 1, the maytansinoid is maytansin itself, which in the unconjugated form has the structure of FIG. 2 (a), may be used to generate the oligo-glycine peptide (Glyₙ) derivative depicted here, which has formed the basis for the immunoligand maytansine conjugates analyzed herein.

FIGS. 2(a)-2(c): Three Maytansinoids that can be used in the context of the present invention. FIG. 2(a): Maytansine, FIG. 2 (b): DM1 ([N²′-deacetyl-N²′-(3-mercapto-1-oxopropyl)-maytansine], FIG. 2(c): DM4 ([N20-deacetyl-N20-(4-mercapto-4-methyl-1-oxo-pentyl)-maytansine].

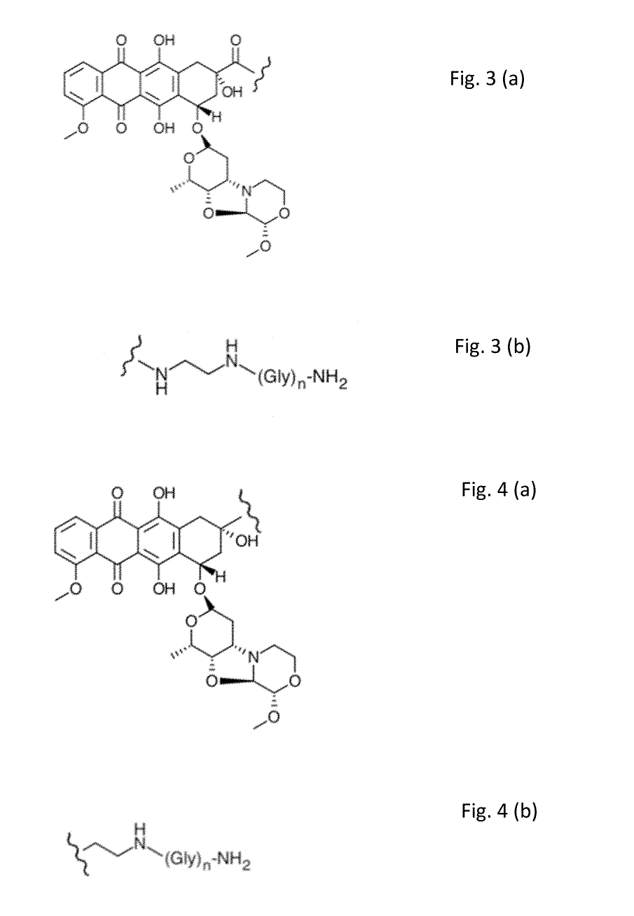

FIG. 3 (a): An anthracycline (PNU) derivative that can be used with the Immunoligand-Toxin-conjugate according to the invention. The derivative may comprise at its wavy line a chemical structure comprising an oligo-glycine peptide (Glyₙ) coupled to said anthracyline derivative in such a way that the oligo-glycine (Glyₙ) peptide has a free amino terminus, and wherein n is an integer between ≥1 and ≤21. The derivative is derived from anthracycline PNU-159682 having the formula (v) as depicted in FIG. 5.

FIG. 3 (b): An oligo-glycine peptide (Glyₙ) is coupled to the anthracyline derivative as seen in FIG. 3 (a) by means of an ethylenediamino linker (EDA), which ethylenediamino linker is coupled to the anthracycline derivative by means of a first amide bond, while it is conjugated to the carboxyterminus of the oligo-glycine peptide by means of a second amide bond, said ethylenediamino linker and oligo-glycine peptide.

FIG. 4 (a): Another anthracycline (PNU) derivative that can be used with the Immunoligand-Toxin-conjugate according to the invention. The derivative may comprise at its wavy line a chemical structure comprising an oligo-glycine peptide (Glyₙ) coupled to said anthracyline derivative in such a way that the oligo-glycine (Glyₙ) peptide has a free amino terminus, and wherein n is an integer between ≥1 and ≤21. The derivative is derived from anthracycline PNU-159682 having the formula (v) as depicted in FIG. 5.

FIG. 4 (b): An oligo-glycine peptide (Glyₙ) is coupled to the anthracyline derivative as seen in FIG. 4 (a) by means of an ethylenediamino linker (EDA), which ethylenediamino linker is coupled to the anthracycline derivative by means of a first amide bond, while it is conjugated to the carboxyterminus of the oligo-glycine peptide by means of a second amide bond, said ethylenediamino linker and oligo-glycine peptide.

FIG. 5: Structure of PNU-159682 as described in the prior art (e.g. WO2009099741, or Quintieri L et al (2005) Clin Cancer Res. 11, 1608-17.

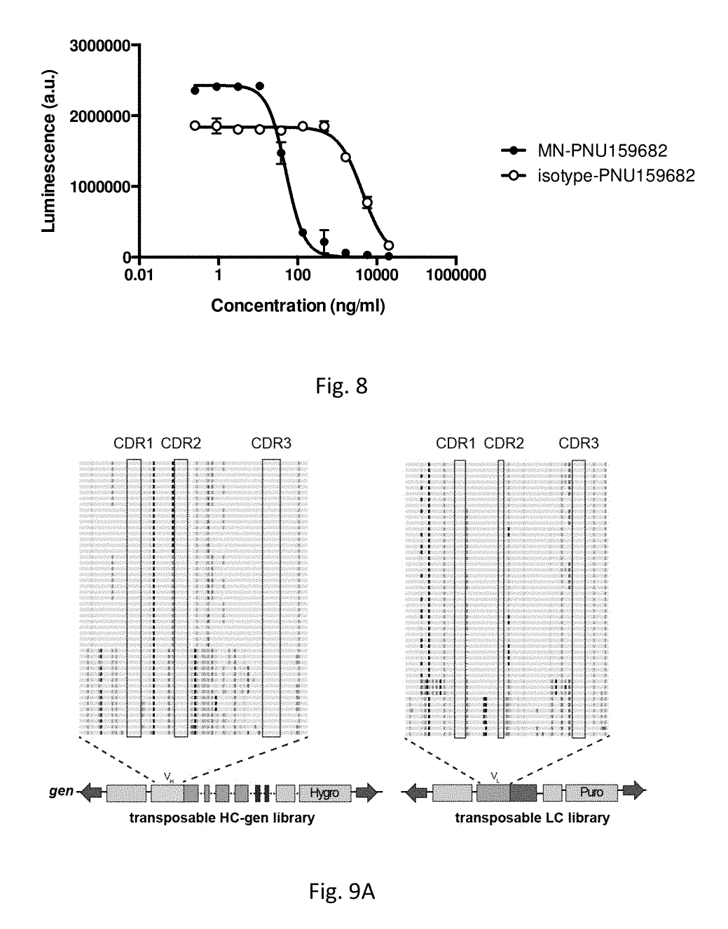

FIG. 6: Structure of PNU-EDA-Gly₅ as utilized for the SMAC-technology conjugation to C-terminally LPETG sortase tagged monoclonal antibodies using sortase enzyme as disclosed in the Examples herein.

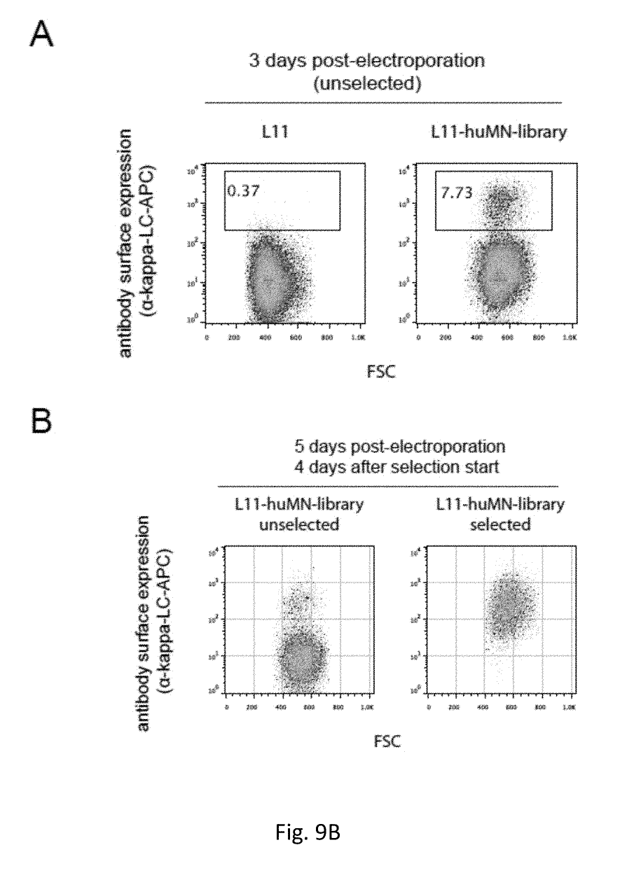

FIG. 7: Schematic drawing of site-specific sortase mediated antibody conjugation (SMAC-technology). The monoclonal antibodies need to be produced with C-terminal LPXTG sortase tags. The toxic payload needs to be produced to contain an oligoglycine peptide stretch (Glyₙ-stretch) with a certain number of glycine (n≥1 and ≤21, preferably n≥3 and ≤10, most preferably n=5) residues in a row. Sortase A enzyme from Staph. aureus specifically recognizes the LPXTG pentapeptide motif and catalyzes the transpeptidation of the oligo-glycine peptide stretch to the threonine-glycine peptide bond of LPXTG, thereby generating a new stabile peptide bond between the threonine and the N-terminal glycine of the oligo-glycine stretch.

FIG. 8: In vitro cell killing of EMT6 cells ectopically expressing mesothelin. EMT6-mesothelin cells were grown in the presence of serial dilutions of an ADC, namely the chimerized mouse mAb MN (Onda et al., Clin Cancer Res 2005; 11(16) Aug. 15, 2005) conjugated to the anthracycline-derivative PNU159682 as payload by means of the sortase mediated conjugation described herein. The benchmark antibody is Trastuzuzmab conjugated to the same toxin. After four days of treatment, viable cells were quantified using a luminescent cell viability assay. Each data point represents the mean of duplicates and error bars represent SD.

FIG. 9A: Schematic representation of the amino acid sequence alignments of humanization libraries. 47 CDR-grafted MN heavy and light chain variable regions were generated by total gene synthesis, mixed and cloned upstream of heavy and light chain constant region coding regions, respectively, as shown. Colored residues represent amino acids different from parental mAbs, grey residues are identical. Complementarity determining regions (CDRs) are indicated according to IMGT numbering.

FIG. 9B: Generation and screening of MN humanization library by Transpo-mAb

(A) Surface antibody expression of cellular huMN library after transposition. A transposable library encompassing 47 HC (genomic variant)×47 LC was electroporated into 3.2E6 cells along with the transposase expression construct using DNA ratios as described in FIG. 3A. To determine transposition efficiency, 1/64 of the total cellular library was cultured without antibiotics for 3 days until transposition was complete, and surface expression was detected by staining with APC-coupled anti-human-kappa-LC. Percentages of surface-expression positive cells are indicated.

(B) Evaluation of selection efficiency. Surface antibody expression after 4 days of selection was determined as described above. Unselected cells were also stained as a control.

(C) Antigen/surface-antibody double staining of selected cellular humanization library for FACS single-cell sort. The selected library (L11-huMN-library) was stained for antigen binding using strep-tagged antigen used at limiting concentration. Bound antigen was detected by fluorophore-conjugated anti-strep-tag antibody. Antibody surface expression was detected using a PE-labelled polyclonal Fc-specific anti-human-IgG antibody, allowing sorting of clones with low surface antibody expression and thus apparently low signal of antigen-binding. A control staining without antigen (-antigen) was included to discriminate true binding from background. Untransposed cells (L11) and cells transposed with parental antibody MN (L11-MN) were stained as negative and positive controls, respectively. Single cells were sorted into 96-well plates according to the representative sorting gate shown in red.

(D) Scatter-plot of single-cell clone supernatants analysed in parallel for antigen-binding and IgG titer by ELISA. Serial dilutions of clonal supernatants of clones grown in 96-well plates were directly used for assessing binding to ELISA plates coated with limiting concentrations of Mesothelin to minimize avidity. IgG levels were determined by sandwich ELISA. EC50 values were calculated using standard curves obtained with known concentrations of parental mAb MN (green). Clone numbers chosen for sequence recovery are indicated (orange).

FIGS. 10A-10D: Sequence recovery and affinities of humanized MN antibodies

FIG. 10A Overview of sequences recovered from top 9 humanized cell clones. Sequences of variable regions were obtained by RT-PCR, cloning into episomal production vectors and sequencing of at least 3 bacterial clones for each cell clone. Numbers of unique sequences found per clone are indicated, as well as numbers of sequences matching the sequences of the designed libraries. Note: Recovered sequences that combined stretches of different library sequences were considered to be artefacts due to PCR-crossover between highly similar strands contained within the library. Only sequences from clones containing library matches for both VH and VL were investigated further (green).

FIG. 10B Deconvolution and validation of recovered sequences. All possible combinations of VH/VL pairs per clone along with the parental pair as a control were transiently transfected into 293T cells. Cell clones and supernatants were analysed for antigen-binding and IgG titer by ELISA as described in FIG. 4D. Ratios of antigen-binding/IgG titers were determined to obtain antigen binding values normalized to IgG content in supernatants (Meso-binding/IgG). Normalized binding is also shown in relation to parental mAb (binding % of parental).

FIG. 10C Isoaffinity plot showing association (kₐ) and dissociation constants (k_{d}) as determined by surface plasmon resonance (SPR). The same supernatants as described in (B) were used for the analysis. Diagonal lines represent equal affinities.

FIG. 10D Table of the same dataset as in (C), showing affinities (KD=k_{d}/kₐ).

FIGS. 11A-11B: Direct functional evaluation of Transpo-mAb-generated single-cell clone supernatants

FIG. 11A Mesothelin expression of cells used for functional evaluation of single-cell clone supernatants generated during Transpo-mAb-based humanization of anti-Mesothelin antibody MN. EMT6 cells stably overexpressing Mesothelin were generated by PiggyBac transposition using a transposable construct containing full length human Mesothelin ORF and a Puromycin selection marker. After transposition, cells were selected using Puromycin and a single-cell clone expressing desired levels of Mesothelin was isolated by FACS. Mesothelin expression of overexpressing clone (EMT6-Meso) and parental cells (EMT6) was analysed by flow cytometry after staining with anti-Mesothelin antibody MN and PE-coupled Fc-specific anti-human-IgG as a secondary antibody.

FIG. 11B Evaluation of Transpo-mAb generated clone supernatants in a secondary antibody-drug-conjugate (2° ADC) cell killing assay. EMT6-Meso target cells were seeded one day before addition of 3.5-fold serial dilutions of clone supernatants containing secreted humanized anti-Mesothelin antibodies. Note: starting concentrations of supernatants were not normalized for IgG content. After 30 min of incubation allowing binding of mAbs to cell surface-expressed target, 2°-ADC (polyclonal anti-human-IgG antibody conjugated with monomethyl-auristatin E (MMAE), an antimitotic, cytotoxic agent) were added and cells were incubated for 3 days. Viable cells were then quantified using a luminescent cell viability assay.

FIG. 12: SPR response curves of humanized MN mAbs. Supernatants of the indicated antibodies were generated by transient expression of 293T cells and affinities were determined using a Biacore T200 instrument. Humanized antibodies were captured using an immobilized anti-human Fcγ-specific antibody. Measurements using four different concentrations of Mesothelin (1:2 serial dilutions starting at 10 nM, with highest concentration measured in duplicate) are shown. Sensorgrams are colored, best-fit curves are shown in black.

FIG. 13: Comparison of generated humanized antibodies alongside clinically approved humanized mAbs with human germline genes. Variable regions of indicated antibodies were subjected to Ig-Blast database search (http://www.ncbi.nlm.nih.gov/igblast/) for the closest human germline sequence each, and sequence identity within framework regions 1, 2 and 3 were determined. Average identity with human germline over all three frameworks was considered as a measure of humanization grade and is shown in percent. For libraries, mean values over all sequences within the library are shown. FDA approval status and sequences of reference humanized antibodies and were retrieved from http://imgt.org/.

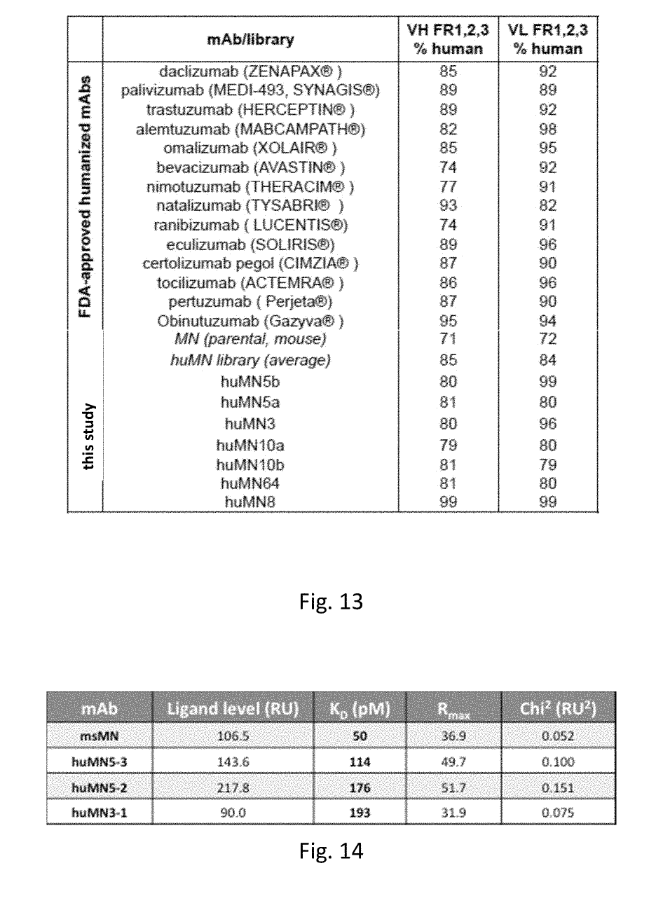

FIG. 14: Determination of huMN affinities. See text for further details.

Sequences:

| SEQ | | | |
|---|---|---|---|
| ID | Sequence | antibody | |
| NO | Type | type | Sequence |
| 1 | CDR1 HC | VH-MN | GYTFTSYW |
| 2 | CDR2 HC | VH-MN | IHPNSDNT |
| 3 | CDR3 HC | VH-MN | AIIITPVVPKFDY |
| 4 | CDR1 LC | VH-MN | HDVGTS |
| 5 | CDR2 LC | VH-MN | WAS |
| 6 | CDR3 LC | VH-MN | QQYSSYPLT |
| 7 | VR HC | VH-MN | QVQLQQPGAELVKPGASMKLSCKASGYTFTSYWMHWVKQRPGQGLEWIGMIHPNSDNTIYYEKFKS |
| | | (parental | KATLTVDKSSSTAYMQLSSLTSEDSAVYYCAIIITPVVPKFDYWGQGTTLTVSS |
| | | mouse) | |
| 8 | VR LC | VH-MN | DIVMTQSHQFMSTSVGDRVSVTCKASHDVGTSVAWYQQKPGQSPKLLIYWASTRHTGVPDRFTGSG |
| | | (parental | SGTDFTLTISNVQSEDLADYFCQQYSSYPLTFGAGTKLELK |
| | | mouse) | |
| 9 | VR HC | VH-MN clone | QVQLQQSGAELVKPGASMKLSCKASGYTFTSYWMHWVRQAPGQGLEWIGAIHPNSDNTIYYQKFKS |
| | | 3-1 | RVTLTVDKSISTAYMQLSSLTSEDTAVYYCAIIITPVVPKFDYWGQGTTVTVSS |
| | | (humanized) | |
| 10 | VR LC | VH-MN clone | DIQMTQSPSFLSASVGDRVTITCRASHDVGTSLAWYQQKPGQAPKLLIYWASTLQTGVPSRFSGSG |
| | | 3-1 | SGTEFTLTISSLQPEDFATYYCQQYSSYPLTFGPGTTVDMK |
| | | (humanized) | |
| 11 | VR HC | VH-MN clone | QVQLQQSGAELVKPGASLKLSCKASGYTFTSYWMHWVRQAPGQGLEWIGAIHPNSDNTIYYQKFKS |
| | | 5-2 | RFTITVDKSTSTAYMQLSSLTSEDTAVYYCAIIITPVVPKFDYWGQGTTVTVSS |
| | | (humanized) | |
| 12 | VR LC | VH-MN clone | DIVMTQSSSFMSASVGDRVSITCKASHDVGTSLAWYQQKPGQSPKLLIYWASTRQTGVPDRFSGSG |
| | | 5-2 | SGTDFTLTISSVQSEDVATYFCQQYSSYPLTFGQGTKLEIK |
| | | (humanized) | |
| 13 | VR HC | VH-MN clone | QVQLQQSGAELVKPGASLKLSCKASGYTFTSYWMHWVRQAPGQGLEWIGAIHPNSDNTIYYQKFKS |
| | | 5-3 | RFTITVDKSTSTAYMQLSSLTSEDTAVYYCAIIITPVVPKFDYWGQGTTVTVSS |
| | | (humanized) | |
| 14 | VR LC | VH-MN clone | DIVMTQSPDSLAVSLGERATITCKSSHDVGTSLAWYQQKPGQPPKLLIYWASTRESGVPDRFSGSG |
| | | 5-3 | SGTDFTLTISSLQAEDVAVYYCQQYSSYPLTFGQGTKLEIK |
| | | (humanized) | |

### INCORPORATION BY REFERENCE

This specification comprises several references to the sortase mediated antibody conjugation (SMAC). This technology is fully disclosed in WO2014140317, content of which is incorporated by reference herein for enabling purposes.

1. A human or humanized antibody, antibody-based binding protein, modified antibody format retaining target binding capacity, or antibody derivative or fragment retaining target binding capacity, which targets Mesothelin (MN).

2. The antibody of claim 1, which comprises at least the 3 CDR sequences:
SEQ ID NO: 1
CDR1 HC
SEQ ID NO: 2
CDR2 HC
SEQ ID NO: 3
CDR3 HC.

3. The antibody of claim 1, which comprises at least the 3 CDR sequences:
SEQ ID NO: 4
CDR1 LC
SEQ ID NO: 5
CDR2 LC
SEQ ID NO: 6
CDR3 LC.

4. The antibody according to claim 1, which comprises at least one heavy chain or light chain variable region sequence that is at least 95% identical to a sequence selected from the group consisting of:
SEQ ID NO: 9
VR HC,
SEQ ID NO: 10
VR LC,
SEQ ID NO: 11
VR HC,
SEQ ID NO: 12
VR LC,
SEQ ID NO: 13
VR HC,
and
SEQ ID NO: 14
VR LC.

5. The antibody according to claim 1, which is humanized from
murine anti Mesothelin antibody VH-MN
and/or which is selected from the group consisting of
VH-MN clone 3-1 (humanized), also called huMN3-1,
VH-MN clone 5-2 (humanized), also called huMN5-2 and
VH-MN clone 5-3 (humanized), also called huMN5-3
and/or antibodies sharing at least 95% amino acid sequence identity with any of the antibodies mentioned above.

6. (canceled)

7. (canceled)

8. The antibody, antibody-based binding protein, modified antibody format, or antibody derivative or fragment of claim 1, wherein the Mesothelin (MN) is human MN.

9. (canceled)

10. An isolated nucleic acid sequence, or a set of isolated nucleic acid sequences, that encodes the antibody, antibody-based binding protein, modified antibody format, or antibody derivative or fragment according to claim 1.

11. A vector comprising at least one nucleic acid sequence according to claim 10.

12. An isolated cell expressing the antibody, antibody-based binding protein, modified antibody format, or antibody derivative or fragment according to claim 1.

13. A method of producing an antibody, antibody-based binding protein, modified antibody format, or antibody derivative or fragment, comprising culturing a cell according to claim 12, and purifying the antibody, antibody-based binding protein, modified antibody format, or antibody derivative or fragment.

14. An Immunoligand-Drug Conjugate having the general formula A-(L)n-(T)m, in which
A is an Immunoligand targeting Mesothelin (MN),
L is a linker,
T is a toxin
and in which n and m are integers between ≥1 and ≤10, and the Immunoligand is the antibody according to claim 1.

15. (canceled)

16. (canceled)

17. The Immunoligand-Drug Conjugate according to claim 14, wherein the linker is at least one selected from the group consisting of
an oligopeptide linker, and
a maleimide linker, optionally comprising cleavable spacers, that may be cleaved by changes in pH, redox potential and/or specific intracellular enzymes.

18. The Immunoligand-Drug Conjugate according to claim 14, wherein the linker comprises an oligopeptide of the sequence selected from the group consisting of LPXSGₙ, LPXAGₙ, LPXTGₙ, LAXTGₙ, LAETGₙ, LPXTAₙ and NPQTGₙ, with n being an integer between ≥1 and ≤21, and X being any amino acid.

19. The Immunoligand-Drug Conjugate according to claim 14, wherein the linker is conjugated to the C-terminus of at least one subdomain of the Immunoligand.

20. The Immunoligand-Drug Conjugate according to claim 14, wherein, prior to conjugation,
the Immunoligand bears a sortase recognition tag used or conjugated to the C-terminus of at least one subdomain thereof, and
the toxin comprises a short glycine stretch with a length of 1-20 glycine residues, preferably with a length of 3 to 5 amino acids.

21. The Immunoligand-Drug Conjugate according to claim 14, wherein the toxin is at least one selected from the group consisting of
maytansinoids,
auristatins,
anthracyclins,
PNU-derived anthracyclins,
calcheamicins,
tubulysins,
duocarmycins,
radioisotopes,
liposomes comprising a toxin payload,
protein toxins,
taxanes, and
pyrrolbenzodiazepines.

22. (canceled)

23. (canceled)

24. A method of producing an Immunoligand-Drug Conjugate according to claim 1, which method comprises the following steps:
a) providing an Immunoligand according to the list set forth in claim 15, which Immunoligand carries a sortase recognition tag,
b) providing one or more toxins carrying an oligoglycine tag, and
c) conjugating the Immunoligand and the toxin by means of sortase-mediated conjugation.

25. A Mesothelin (MN) specific chimeric antigen receptor (CAR), comprising at least one antibody, antibody-based binding protein, modified antibody format, or antibody derivative or fragment according to claim 1.

26. A cell comprising the chimeric antigen receptor according to claim 25, which cell is an engineered T-cell.

27. A method of treating a patient that is suffering from, at risk of developing, and/or being diagnosed for a neoplastic disease, comprising administering to the patient an effective amount of the antibody, antibody-based binding protein, modified antibody format retaining target binding capacity, or antibody derivative or fragment according to claim 1.

28. (canceled)

29. A pharmaceutical composition comprising the antibody, antibody-based binding protein, modified antibody format retaining target binding capacity, or antibody derivative or fragment according to claim 1, together with one or more pharmaceutically acceptable ingredients.

30. A method of killing or inhibiting the growth of a cell expressing Mesothelin (MN) in vitro or in a patient, which method comprises administering to the cell or to the subject a pharmaceutically effective amount of the antibody, antibody-based binding protein, modified antibody format retaining target binding capacity, or antibody derivative or fragment according to claim 1.

31. (canceled)

32. (canceled)
